(19) United States
(12) Patent Application Publication
(10) Pub. No.: US 2010/0234390 A1
(43) Pub. Date: Sep. 16, 2010

(54) NOVEL COMPOUND AND THEIR USE

(76) Inventors: Simi Pushpan, Tanil Nadu (IN); Uma Ramachandran, Tamil Nadu (IN); Mrinalkanti Kundu, Tamil Nadu (IN)

Correspondence Address: LADAS & PARRY LLP, 26 WEST 61ST STREET, NEW YORK, NY, 10023, US

(21) Appl. No.: 12/665,945

(22) PCT Filed: Jun. 20, 2008
(86) PCT No.: PCT/IB08/01620

§ 371 Date: Jun. 4, 2010

(30) Foreign Application Priority Data
Jun. 22, 2007 (IN) 129/CHE/2007
Mar. 5, 2008 (IN) 560/CHE/2008

(51) Int. Cl. A61K 31/497; C07D 413/10; C07D 241/24; C07D 401/14; A61P 31/04

(52) U.S. Cl. 514/252.11; 544/369; 514/254.02; 544/357; 544/364; 514/253.09

(57) ABSTRACT

The present invention provides novel compounds of the general formula (I), their derivatives, analogs, tautomeric forms, stereoisomers, polymorphs, hydrates, solvates, pharmaceutically acceptable salts and compositions, metabolites and prodrugs thereof. The novel compounds are useful as antibacterial agents in the treatment of conditions such as nosocomial pneumonia, community acquired pneumonia, infections caused by vancomycin resistant enterococci (VRE), methicillin resistant Staphylococcus aureus (MRSA), Heamophilus influenzae (HI) and penicillin resistant Streptococcus pneumoniae (PRSP). The compounds of the present invention are effective against a number of human or animal pathogens including VRE, PRSP, HI and MRSA.

### FIELD

Described are novel compounds of the general formula (I), their derivatives, analogs, tautomeric forms, stereoisomers, polymorphs, hydrates, solvates, pharmaceutically acceptable salts and compositions, metabolites and prodrugs thereof. The present invention more particularly provides novel oxazolidinone derivatives of the general formula (I).

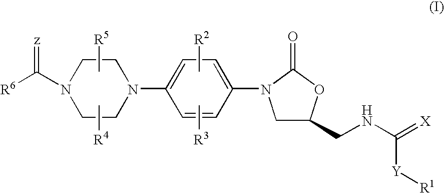

The present invention also provides a process for the preparation of the above said novel oxazolidinone derivatives of the formula (I), their derivatives, analogs, tautomeric forms, stereoisomers, polymorphs, hydrates, solvates, pharmaceutically acceptable salts and compositions, metabolites and prodrugs thereof. This invention also relates to intermediates useful in the preparation of such compounds.

The novel oxazolidinone derivatives of the present invention are useful as antibacterial agents in the treatment of conditions such as nosocomial pneumonia, community acquired pneumonia, infections caused by vancomycin resistant enterococci (VRE), methicillin resistant Staphylococcus aureus (MRSA), Heamophilus influenzae (HI) and penicillin resistant Streptococcus pneumoniae (PRSP). The compounds of the present invention are effective against a number of human or animal pathogens including PRSP, VRE, MRSA and HI.

### BACKGROUND

Several oxazolidinone derivatives have been reported in the literature some of the relevant ones are given here:

WO 97/09328 discloses compounds of formula (II)

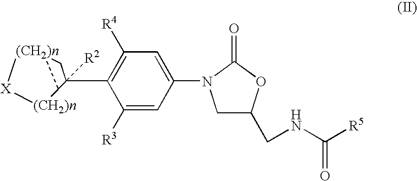

in which X is NR¹, S(O)_{g} or O; R¹ is a hydrogen, (C₁-C₆)alkyl optionally substituted with one or more OH, CN, or halo or R¹ is —(CH₂)ₕ-aryl, —COR¹⁻¹, COOR¹⁻², —CO—(CH₂)ₕ—COR¹⁻¹, (C₁-C₆)alkylsulfonyl, —SO₂—(CH₂)ₕ-aryl or —(CO)₁-Het; R² is hydrogen, (C₁-C₆)alkyl, —(CH₂)ₕ-aryl or halo; R³ and R⁴ are the same or different and are hydrogen or halo; R⁵ is hydrogen, (C₁-C₁₂)alkyl optionally substituted with one or more halo, (C₃-C₁₂)cycloalkyl, (C₁-C₆)alkoxy; g is 0, 1 or 2; h is 1, 2, 3 or 4; i is 0 or 1; m is 0, 1, 2, 3, 4, or 5; n is 0, 1, 2, 3, 4 or 5.

WO 97/30995 discloses compounds of formula (III)

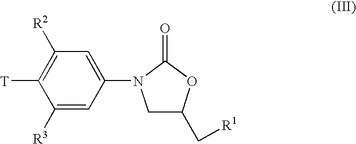

wherein T is of the formula

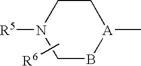

wherein R¹ is chloro, fluoro, (C₁-C₄)alkanesulfonyloxy, azido, (C₁-C₄)alkoxy, (C₁-C₄)alkylthio, (C₁-C₄)alkylaminocarbonyloxy; or of the formula —NHC(═O)R^{b} wherein R^{b} is hydrogen, (C₁-C₄)alkoxy, amino, chloromethyl, dichloromethyl, cyanomethyl, methoxymethyl, acetylmethyl, methylamino, dimethylamino or (C₁-C₄)alkyl; or of the formula —NHS(O)ₙ(C₁-C₄)alkyl where n is 0, 1 or 2; R² and R³ are independently hydrogen or fluoro; >A-B— is >CH—C₁₋₁₂; R⁶ is (C₁-C₄)alkyl, (C₁-C₄alkanoylamino(C₁-C₄)alkyl, hydroxy(C₁-C₄)alkyl, carboxy, (C₁-C₄)alkoxycarbonyl, Ar-oxymethyl, Ar-thiomethyl (where Ar is as defined in the specification) or independently as defined for R⁵ excluding hydrogen; R⁵ is of the formula R¹⁰CO—, R¹⁰SO₂—, R¹⁰CS—, where R¹⁰ is Ar.

U.S. Pat. No. 5,922,708 discloses compounds of formula (IV)

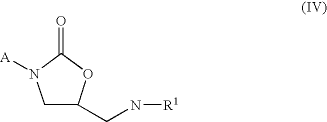

in which R¹ is a radical of the formula D-R², —CO—R³ or —CO—NR⁴R⁵, wherein D is the CO₂ or SO₂ group, R² is phenyl or linear or branched alkyl having up to 7 carbon atoms, R³ is trifluoromethyl or linear or branched alkyl having up to 6 carbon atoms which is substituted by halogen or trifluoromethyl, and R⁴ and R⁵ are identical or different and are hydrogen, phenyl or linear or branched alkyl having up to 5 carbon atoms; A is a 6-membered aromatic heterocycle having at least one nitrogen atom and directly bonded via a carbon atom, or a 6-membered bicyclic or tricyclic aromatic radical having at least one nitrogen-containing ring and directly bonded via a carbon atom, or β-carbolin-3-yl or indolizinyl directly bonded via the 6-membered ring, or a 5-membered aromatic heterocycle having up to 3 heteroatoms from the group S, N and/or O and directly bonded via a carbon atom, which heterocycle can additionally have a fused benzene or naphthyl ring, all the rings optionally being substituted in each case by up to 3 identical or different substituents selected from carboxyl, halogen, cyano, mercapto, formyl, trifluoromethyl, nitro, linear or branched alkoxy, alkoxycarbonyl, alkylthio or acyl, each of which has up to 6 carbon atoms, and linear or branched alkyl having up to 6 carbon atoms, which in turn can be substituted by hydroxyl, by linear or branched alkoxy or acyl, each of which has up to 5 carbon atoms, or by a group of the formula —NR⁶, R⁷, wherein R⁶ and R⁷ are identical or different and are hydrogen, cycloalkyl having 3 to 6 carbon atoms, linear or branched alkyl having up to 5 carbon atoms or phenyl, or together with the nitrogen atom, form a 5- or 6-membered saturated heterocycle optionally having another heteroatom from the group N, S and/or O, which heterocycle in turn can optionally be substituted, also on another nitrogen atom, by linear or branched alkyl or acyl, each of which has up to 3 carbon atoms etc.

EP 1130016 discloses thiocarbamic acid derivatives, which are useful as antimicrobial agents compounds of formula (V)

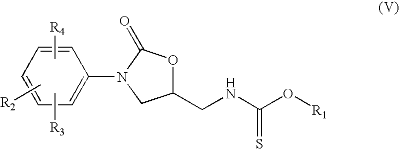

wherein R₁ represents an alkyl group which may be substituted, or a cycloalkyl group which may be substituted; and R₂, R₃ and R₄ independently represent hydrogen atom, a halogen atom, substituted or unsubstituted an alkyl, alkoxyl, amino, alkanoyl, cycloalkyloxy or a saturated heterocyclic group.

US 2003119817 discloses oxazolidinone derivatives as potential antimicrobials which are useful as antimicrobial agents compounds of formula (VI)

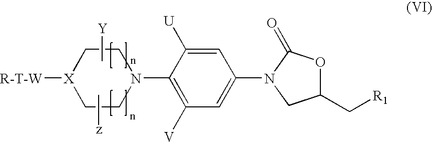

wherein R₁ is selected from the group consisting of —NHC(═O)R₂, —NR2C(═S)R₃; wherein R₂ is hydrogen, C₁₋₁₂ alkyl, C₃₋₁₂ cycloalkyl, alkoxy, C₁₋₆ alkyl substituted with one or more of F, Cl, Br, I or OH; N(R₃, R₄); R₃ represents hydrogen, alkyl, cycloalkyl and alkoxy; U and V are hydrogen or fluoro; X is CH, CH—S, CH—O and N; n is an integer in the range from 0 to 3; W is selected from the group CH₂, CO, CH₂NH, —NHCH₂, S, CH₂(CO), N(R₁₁) wherein R₁₁ is hydrogen, optionally substituted C₁₋₁₂ alkyl, C₃₋₁₂ cycloalkyl, C₁₋₆ alkoxy, C₁₋₆ alkyl, aryl or heteroaryl; T is five to seven membered heterocyclic ring, aryl, substituted aryl, bound to the ring C with a linker W and the heterocyclic and aryl rings are further substituted by a group represented by R, R is selected from the group consisting of alkyl (C₁₋₆), halogen, CN, COR₅, N(R₆,R₇), CON(R₆R₇), CH₂NO₂, NO₂.

WO 2005003087 discloses oxazolidinone derivatives as antibacterial agents of formula (VII)

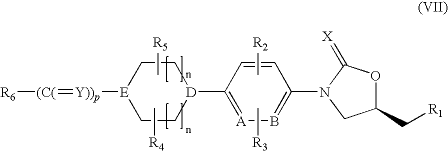

wherein, R₁ represents the formula —NHC(═Z)R₉ wherein Z represents O or S, R₉ is hydrogen, substituted or unsubstituted groups selected from (C₁-C₆) alkyl, (C₁-C₆) alkoxy, aryl, X and Y represent oxygen or sulfur; A and B are different and represent CH or N; R₂ and R₃ may be same or different and independently represent hydrogen, halogen, hydroxy, alkyl and alkoxy; n is an integer of 0 or 1; m is an integer in the range of 1 to 4; 1) represents CH or N; E represents CH or N; R₄ and R₅ represent hydrogen, cyano, nitro, amino, halogen and hydroxyl; p is an integer of 1; R₆ represents a substituted or unsubstituted groups selected from aryl, cycloalkyl, aralkyl, heteroaryl, heteroaralkyl, heteroaralkenyl, heterocyclyl, heterocycloalkyl, heterocycloalkenyl.

WO 20000332599 discloses oxazolidinone antibacterial agents having a thiocarbonyl functionality of formula (VIII)

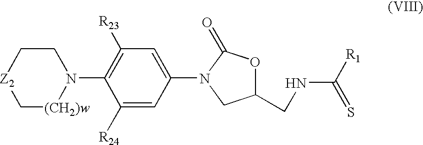

Wherein R₁ is H, NH₂, NHalkylC₁₋₄; N(alkylC₁₋₄)₂; alkylC₁₋₄; OalkylC₁₋₄; SalkylC₁₋₄; alkylC₁₋₄, R₂₃ and R₂₄ are same or different and are H, F, Cl, C₁₋₂ alkyl, CN, OH, nitro and amino; W represents 0, 1, 2 or 3; Z₂ is —O₂S—, —O—, —N(R₁₀₇)—, —OS—, or —S—; R₁₀₇ is R₁₀₂O—C(R₁₁₀)(R₁₁₀)(R₁₁₁)—C(O)—, R₁₀₃O—C(O)—, R₁₀₈—C(O)—, R₁₀₉—SO₂—NC—CH₂—, F₂CHCH₂—, R₁₅₀R₁₅₁NSO₂—; wherein R₁₀₂ is H, —CH₃, phenyl-CH₂—, or CH₃C(O); each of R₁₁₀ and R₁₁₁ is selected from H or CH₃.

### OBJECTIVE

We have focused our research to identify novel oxazolidinone derivatives, which will be effective against drug resistant bacteria. Apart from this we have incorporated the structural features to enhance its activity towards gram-negative microorganisms like Heamophilus influenzae and Moraxella catarrhalis. Our sustained efforts have resulted in novel oxazolidinone derivatives of the formula (I). The novel oxazolidinone derivatives of the present invention are useful as antibacterial agents, useful in the treatment of conditions such as nosocomial pneumonia, community acquired pneumonia, infections caused by vancomycin resistant enterococci (VRE), methicillin resistant Staphylococcus aureus (MRSA), Heamophilus influenzae (HI) and penicillin resistant Streptococcus pneumoniae (PRSP). The compounds of the present invention are effective against a number of human or animal pathogens including VRE, PRSP, HI and MRSA.

### SUMMARY

The present invention relates to novel oxazolidinone derivatives of the formula (I)

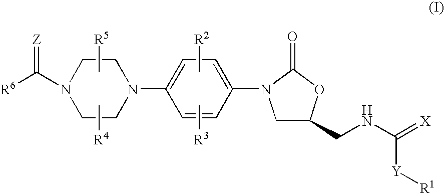

their derivatives, analogs, tautomeric forms, stereoisomers, polymorphs, hydrates, solvates, pharmaceutically acceptable salts and compositions, metabolites and prodrugs thereof, wherein X and Y independently represent oxygen or sulfur; Z represent oxygen; sulphur or N(O)ₙR⁷, wherein R⁷ represents substituted or unsubstituted groups selected from hydrogen; hydroxyl; cyano; alkyl; cycloalkyl; haloalkyl; aryl; aralkyl; acyl; benzoyl; thioacyl; arylsulfonyl; aralkylsulfonyl; heteroaryl and heterocyclyl; R¹ represents hydrogen; substituted or unsubstituted groups selected from (C₁-C₆)alkyl; haloalkyl; (C₃-C₆)cycloalkyl; alkylamine; aryl; aralkyl; heterocyclyl; heteroaryl; alkylsulfonyl; arylsulfonyl and aralkylsulfonyl; R² and R³ may be same or different and independently represent hydrogen; halogen; hydroxyl; alkyl and alkoxy groups; R⁴ and R⁵ may be same or different and independently represent hydrogen; cyano; nitro; amino; halogen; hydroxyl; substituted or unsubstituted groups selected from (C₁-C₆)alkyl; haloalkyl; (C₁-C₆)alkoxy; (C₁-C₆)alkylthio; (C₃-C₆)cycloalkyl or either of R⁴ or R⁵ represent an oxo or thiooxo group, substituted or unsubstituted benzyl group; R⁶ represents a substituted or unsubstituted alkyl; haloalkyl; cycloalkyl; mono or dihydroxyalkyloxymethyl; alkylamine; aryl; aralkyl; arylalkenyl; bezyloxyalkyl; heteroaryl; heteroaralkyl; heteroarylalkenyl; heterocyclyl; heterocyclylalkyl; substituted or, unsubstituted groups selected from TR⁸, wherein T represents O or S; R⁸ represents substituted or unsubstituted groups selected from (C₁-C₆)alkyl; haloalkyl; (C₃-C₆)cycloalkyl; alkylamine; aryl; aralkyl; heterocyclyl; heteroaryl; alkylsulfonyl; arylsulfonyl and aralkylsulfonyl; n is an integer of 0 or 1.

### DETAILED DESCRIPTION

The present invention relates to novel oxazolidinone derivatives of the formula (I),

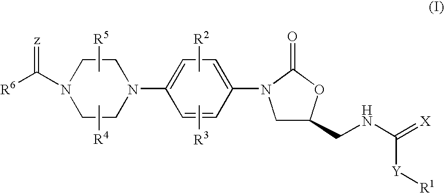

their derivatives, analogs, tautomeric forms, stereoisomers, polymorphs, hydrates, solvates, pharmaceutically acceptable salts and compositions, metabolites and prodrugs thereof, wherein, suitable groups represented by R¹ are selected from hydrogen; (C₁-C₆)alkyl groups such as methyl, ethyl, n-propyl, isopropyl, n-butyl, isobutyl, t-butyl, n-pentyl, isopentyl, hexyl and the like, which may be substituted; haloalkyl groups such as chloromethyl, chloroethyl, trifluoromethyl, trifluoroethyl, dichloromethyl, dichloroethyl and the like, which may be substituted; (C₃-C₆)cycloalkyl groups such as cyclopropyl, cyclobutyl, cyclopentyl and cyclohexyl, which may be substituted; alkylamine groups such as NH₂CH₂—, NH₂C₂H₄—, NH₂C₃H₆—, NH₂C₆H₁₂— and the like, which may be substituted; aryl groups such as phenyl, naphthyl and the like, which may be substituted; aralkyl groups such as phenylmethyl, phenylethyl, naphthylmethyl, naphthylethyl and the like, which may be substituted; heterocyclyl groups such as pyrrolidinyl, morpholinyl, thiomorpholinyl, piperidinyl, piperazinyl and the like; heteroaryl groups such as pyridyl, thienyl, furyl, pyrrolyl, oxazolyl, thiazolyl, imidazolyl, isooxazolyl, oxadiazolyl, triazolyl, thiadiazolyl, tetrazolyl, pyrimidinyl, pyrazinyl, pyridazinyl, benzopyranyl, benzofuranyl, benzimidazolyl, benzoxazolyl, benzothiazolyl, benzopyrrolyl, benzoxadiazolyl, benzothiadiazolyl and the like, which may be substituted; alkylsulfonyl; arylsulfonyl and aralkylsulfonyl, which may be substituted.

Suitable groups represented by R² and R³ are selected from hydrogen; halogen atoms such as fluorine, chlorine, bromine or iodine; hydroxyl; (C₁-C₆) alkyl groups such as methyl, ethyl, n-propyl, isopropyl, n-butyl, isobutyl, t-butyl, n-pentyl, isopentyl, hexyl and the like, which may be substituted; (C₁-C₆) alkoxy groups such as methoxy, ethoxy, n-propoxy, isopropoxy and the like, which may be substituted.

Suitable groups represented by R⁴ and R⁵ are selected from hydrogen; cyano; nitro; amino; halogen; hydroxyl; (C₁-C₆) alkyl groups such as methyl, ethyl, n-propyl, isopropyl, n-butyl, isobutyl, t-butyl, n-pentyl, isopentyl, hexyl and the like, which may be substituted; haloalkyl such as chloromethyl, chloroethyl, trifluoromethyl, trifluoroethyl, dichloromethyl, dichloroethyl and the like, which may be substituted; (C₁-C₆) alkoxy groups such as methoxy, ethoxy, n-propoxy, isopropoxy and the like, which may be substituted; (C₁-C₆)alkylthio groups such as methylthio, ethylthio, n-propylthio, iso-propylthio and the like, which may be substituted; (C₃-C₆)cycloalkyl groups such as cyclopropyl, cyclobutyl, cyclopentyl, cyclohexyl and the like, which may be substituted or either of R⁴ or R⁵ represent an oxo or thioxo group; substituted or unsubstituted benzyl group.

Suitable groups represented by R⁶ are selected from a (C₁-C₆) alkyl groups such as methyl, ethyl, n-propyl, isopropyl, n-butyl, isobutyl, t-butyl, n-pentyl, isopentyl, hexyl and the like, which may be substituted; mono or dihydroxyalkyloxymethyl groups like HO—CH₂—CH₂-O—CH₂—, HO—CH₂—CH(OH)—CH₂—O—CH₂—; haloalkyl such as chloromethyl, chloroethyl, trifluoromethyl, trifluoroethyl, dichloromethyl, dichloroethyl and the like, which may be substituted; (C₃-C₆) cycloalkyl groups such as cyclopropyl, cyclobutyl, cyclopentyl, and cyclohexyl, which may be substituted; alkylamine groups such as NH₂CH₂—, NH₂C₂H₄—, NH₂C₃H₆—, NH₂C₆H₁₂— and the like, which may be substituted; aryl groups such as phenyl, naphthyl and the like, which may be substituted; aralkyl groups such as phenylmethyl, phenylethyl, naphthylmethyl, naphthylethyl and the like, which may be substituted; arylalkenyl groups such as benzene-prop-1-enyl and the like which may be substituted; benzyloxyalkyl such as benzyloxymethyl, benzyloxyethyl, benzyloxypropyl and the like, which may be substituted; heteroaryl groups such as pyridyl, thienyl, furyl, pyrrolyl, oxazolyl, thiazolyl, imidazolyl, isooxazolyl, oxadiazolyl, triazolyl, thiadiazolyl, tetrazolyl, pyrimidinyl, pyrazinyl, pyridazinyl, quinolinyl, quinoxalinyl, indolyl, indazolyl, benzopyranyl, benzofuranyl, benzimidazolyl, benzoxazolyl, benzothiazolyl, benzopyrrolyl, benzoxadiazolyl, benzothiadiazolyl and the like, which may be substituted; heteroaralkyl such as pyridylmethyl, indolylmethyl, triazolylmethyl, tetrazolylmethyl and the like, which may be substituted; heteroarylalkenyl such as furan-ethen-1-yl, furan-prop-1-enyl and the like, which may be substituted; heterocyclyl groups such as pyrrolidinyl, morpholinyl, thiomorpholinyl, thiazolidinyl, piperidinyl, piperazinyl and the like, which may be substituted; substituted or unsubstituted groups selected from TR⁸, wherein T represents O or S; R⁸ represents substituted or unsubstituted groups selected from (C₁-C₆) alkyl groups such as methyl, ethyl. n-propyl, isopropyl, n-butyl, isobutyl, t-butyl, n-pentyl, isopentyl, hexyl and the like, which may be substituted; haloalkyl such as chloromethyl, chloroethyl, trifluoromethyl, trifluoroethyl, dichloromethyl, dichloroethyl and the like, which may be substituted; (C₃-C₆) cycloalkyl groups such as cyclopropyl, cyclobutyl, cyclopentyl and cyclohexyl, which may be substituted; alkylamine groups such as NH₂CH₂—, NH₂C₂H₄—, NH₂C₃H₆—, NH₂C₆H₁₂— and the like; which may be substituted; aryl groups such as phenyl, naphthyl and the like, which may be substituted; aralkyl groups such as phenylmethyl, phenylethyl, naphthylmethyl, naphthylethyl and the like, which may be substituted; heterocyclyl groups such as pyrrolidinyl, morpholinyl, thiomorpholinyl, piperidinyl, piperazinyl and the like, which may be substituted; heteroaryl groups such as pyridyl, thienyl, furyl, pyrrolyl, oxazolyl, thiazolyl, imidazolyl, isooxazolyl, oxadiazolyl, triazolyl, thiadiazolyl, tetrazolyl, pyrimidinyl, pyrazinyl, pyridazinyl, benzopyranyl, benzofuranyl, benzimidazolyl, benzoxazolyl, benzothiazolyl, benzopyrrolyl, benzoxadiazolyl, benzothiadiazolyl and the like, which may be substituted; alkylsulfonyl; arylsulfonyl; aralkylsulfonyl, which may be substituted.

Wherein X and Y independently represent oxygen or sulfur; Z represent oxygen or sulphur or N(O)ₙR⁷, wherein suitable groups represented by R⁷ are selected from hydrogen; hydroxyl; substituted or unsubstituted linear or branched (C₁-C₆)alkyl groups such as methyl, ethyl, n-propyl, isopropyl, n-butyl, isobutyl, t-butyl, n-pentyl, hexyl and the like, which may be substituted; (C₃-C₆) cycloalkyl groups such as cyclopropyl, cyclobutyl, cyclopentyl, cyclohexyl and the like, which may be substituted; haloalkyl groups such as chloromethyl, chloroethyl, trifluoromethyl, trifluoroethyl, dichloromethyl, dichloroethyl and the like, which may be substituted; aryl groups such as phenyl, naphthyl and the like, the aryl groups may be substituted; aralkyl groups such as phenylmethyl, phenylethyl, naphthylmethyl, naphthylethyl and the like, the aralkyl groups may be substituted; acyl groups such as —C(═O)CH₃, —C(═O)C₂H₅, —C(═O)C₃H₇, —C(═O)C₆H₁₃, benzoyl and the like, the acyl groups may be substituted; thioacyl groups such as —C(═S)CH₃, —C(═S)C₂H₅, —C(═S)C₃H₇, —C(═S)C₆H₁₃ and the like, the thioacyl group may be substituted; alkylsulfonyl groups such as methylsulfonyl, ethylsulfonyl, n-propylsulfonyl, iso-propylsulfonyl and the like, which may be substituted; arylsulfonyl groups such as phenylsulfonyl, naphthylsulfonyl and the like, which may be substituted; aralkylsulfonyl groups such as phenylmethylsulfonyl, phenylethylsulfonyl, naphthylmethylsulfonyl, naphthylethylsulfonyl and the like, which may be substituted; heteroaryl groups such as pyridyl, thienyl, furyl, pyrrolyl, oxazolyl, thiazolyl, imidazolyl, isooxazolyl, oxadiazolyl, triazolyl, thiadiazolyl, tetrazolyl, pyrimidinyl, pyrazinyl, pyridazinyl, benzopyranyl, benzofuranyl, benzimidazolyl, benzoxazolyl, benzothiazolyl, benzopyrrolyl, benzoxadiazofyl, benzothiadiazolyl and the like, which may be substituted; heterocyclyl groups such as pyrrolidinyl, morpholinyl, thiomorpholinyl, piperidinyl, piperazinyl, and the like, which may be substituted.

n is an integer of 0 or 1.

The substituents on any of the groups represented by R¹, R², R³, R⁴, R⁵, R⁶, R⁷, and R⁸ are selected from one or more groups represented by halogen; hydroxy; formyl; nitro; cyano; azido; amino; alkyl; aryl; alkylamino; alkylsulfonylamino; aralkoxy; arylthio; alkylaminocarbonyl; haloalkyl; alkylthio; acylamino; alkoxy; acyl; cycloalkylacyl; heteroarylacyl; N-hydroxyimidamide; carboxylic acid and its derivatives such as esters and amides and these substituents are as defined above. The albove substituents, which in turn are further substituted by the groups, selected from alkyl; halogen; amino; nitro; hydroxy and the like.

Pharmaceutically acceptable salts of the present invention include alkali metals like Li, Na, and K, alkaline earth metals like Ca and Mg, salts of organic bases such as diethanolamine, α-phenylethylamine, benzylamine, piperidine, morpholine, pyridine, hydroxyethylpyrrolidine, hydroxyethylpiperidine, choline and the like, ammonium or substituted ammonium salts, aluminum salts. Salts also include amino acid salts such as glycine, alanine, cystine, cysteine, lysine, arginine, phenylalanine, guanidine etc. Salts may include acid addition salts where appropriate which are, sulphates, nitrates, phosphates, perchlorates, borates, hydrohalides, acetates, tartrates, maleates, citrates, succinates, palmoates, methanesulphonates, tosylates, benzoates, salicylates, hydroxynaphthoates, benzenesulfonates, ascorbates, glycerophosphates, ketoglutarates and the like. Pharmaceutically acceptable solvates may be hydrates or comprising other solvents of crystallization such as alcohols.

Representative compounds according to the present invention include:
1. (S)—O-Methyl(3-(4-(4-(benzofuran-2-carbonyl)piperazin-1-yl)-3-fluorophenyl)-2-oxooxazolidin-5-yl)methylearbamothioate;
2. (S)—O-Methyl(3-(3-fluoro-4-(4-nicotinoylpiperazin-1-yl)phenyl)-2-oxooxazolidin-5-yl)methylcarbamothioate;
3. (S)—O-Methyl(3-(3-fluoro-4-(4-(pyrazine-2-carbonyl)piperazin-1-yl)phenyl)-2-oxo oxazolidin-5-yl)methylcarbamothioate;
4. (S)—O-Methyl(3-(3-fluoro-4-(4-(3,4,5-trimethoxybenzoyl)piperazin-1-yl)phenyl)-2-oxooxazolidin-5-yl)methylcarbamothioate;
5. (S)—O-Methyl (3-(4-(4-(4-acetamidobenzoyl)piperazin-1-yl)-3-fluorophenyl)-2-oxooxazolidin-5-yl)methylcarbamothioate;
6. (S)—O-Methyl (3-(3-fluoro-4-(4-(5-methylpyrazine-2-carbonyl)piperazin-1-yl)phenyl)-2-oxooxazolidin-5-yl)methylcarbamothioate;
7. (S)—O-Methyl(3-(3-fluoro-4-(4-(5-nitrofuran-2-carbonyl)piperazin-1-yl)phenyl)-2-oxooxazolidin-5-yl)methylcarbamothioate;
8. (S)—O-Methyl(3-(4-(4-(6-cyanonicotinoyl)piperazin-1-yl)-3-fluorophenyl)-2-oxo oxazolidin-5-yl)methylcarbamothioate;
9. (S)—O-Methyl(3-(3-fluoro-4-(4-(1-methyl-pyrrole-2-carbonyl)piperazin-1-yl)phenyl)-2-oxooxazolidin-5-yl)methylcarbamothioate;
10. (S)—O-Methyl(3-(3-fluoro-4-(4-isonicotinoylpiperazin-1-yl)phenyl)-2-oxooxazolidin-5-yl)methylcarbamothioate;
11. (S)—O-Methyl(3-(3-fluoro-4-(4-(quinoline-3-carbonyl)piperazin-1-yl)phenyl)-2-oxooxazolidin-5-yl)methylcarbamothioate;
12. (S)—O-Methyl (3-(4-(4-(6-chloronicotinoyl)piperazin-1-yl)-3-fluorophenyl)-2-oxooxazolidin-5-yl)methylcarbamothioate;
13. (S)—O-Methyl(3-(4-(4-(1H-indole-2-carbonyl)piperazin-1-yl)-3-fluorophenyl)-2-oxooxazolidin-5-yl)methylcarbamothioate;
14. (5)—O-Methyl (3-(3-fluoro-4-(4-(2-fluoronicotinoyl)piperazin-1-yl)phenyl)-2-oxooxazolidin-5-yl)methylcarbamothioate;
15. (S)—O-Methyl (3-(3-fluoro-4-(4-(5-fluoro-1H-indole-2-carbonyl)piperazin-1-yl)phenyl)-2-oxooxazolidin-5-yl)methylcarbamothioate;
16. (S)—O-Methyl (3-(4-(4-(4-chloronicotinoyl)piperazin-1-yl)-3-fluorophenyl)-2-oxo oxazolidin-5-yl)methylcarbamothioate;
17. (S)—O-Methyl (3-(3-fluoro-4-(4-(6-methylnicotinoyl)piperazin-1-yl)phenyl)-2-oxooxazolidin-5-yl)methylcarbamothioate;
18. (S)—O-Methyl (3-(4-(4-(1H-imidazole-4-carbonyl)piperazin-1-yl)-3-fluorophenyl)-2-oxooxazolidin-5-yl)methylcarbamothioate;
19. (S)—O-Methyl(3-(4-(4-(1-cyanocyclopropanecarbonyl)piperazin-1-yl)-3-fluoro phenyl)-2-oxooxazolidin-5-yl)methylcarbamothioate;
20. (S)—O-Methyl(3-(4-(4-(5-bromonicotinoyl)piperazin-1-yl)-3-fluorophenyl)-2-oxo oxazolidin-5-yl)methylcarbamothioate;
21. (S)—O-Methyl(3-(3-fluoro-4-(4-(quinoxaline-2-carbonyl)piperazin-1-yl)phenyl)-2-oxooxazolidin-5-yl)methylcarbamothioate;
22. (S)—O-Methyl(3-(3-fluoro-4-(4-(6-(trifluoromethyl)nicotinoyl)piperazin-1-yl)phenyl)-2-oxooxazolidin-5-yl)methylcarbamothioate;
23. (S)—O-Methyl (3-(3-fluoro-4-(4-(1-p-tolylcyclopropanecarbonyl)piperazin-1-yl)phenyl)-2-oxooxazolidin-5-yl)methylcarbamothioate;
24. (S)—O-Methyl(3-(4-(4-(2,6-dichloro-5-fluoronicotinoyl)piperazin-1-yl)-3-fluorophenyl)-2-oxooxazolidin-5-yl)methylcarbamothioate;
25. (S)—O-Methyl(3-(4-(4-(1H-indazole-3-carbonyl)piperazin-1-yl)-3-fluorophenyl)-2-oxooxazolidin-5-yl)methylcarbamothioate;
26. (S)—O-Methyl(3-(3-fluoro-4-(4-(6-hydroxynicotinoyl)piperazin-1-yl)phenyl)-2-oxooxazolidin-5-yl)methylcarbamothioate;
27. (S)—O-Methyl(3-(4-(4-(benzo[d]thiazole-6-carbonyl)piperazin-1-yl)-3-fluorophenyl)-2-oxooxazolidin-5-yl)methylcarbamothioate;
28. (S)—O-Methyl(3-(3-fluoro-4-(4-(thiazolidine-2-carbonyl)piperazin-1-yl)phenyl)-2-oxooxazolidin-5-yl)methylcarbamothioate;
29. (S)—O-Methyl(3-(4-(4-(1H-benzo[d]imidazole-5-carbonyl)piperazin-1-yl)-3-fluoro phenyl)-2-oxooxazolidin-5-yl)methylcarbamothioate;
30. (S)—O-Methyl (3-(4-(4-(5-chlorobenzofuran-2-carbonyl)piperazin-1-yl)-3-fluoro phenyl)-2-oxooxazolidin-5-yl)methylcarbamothioate;
31. (S)—O-Methyl (3-(3-fluoro-4-(4-((R)-thiazolidine-4-carbonyl)piperazin-1-yl)phenyl)-2-oxooxazolidin-5-yl)methylcarbamothioate;
32. (S)—O-M ethyl (3-(3-fluoro-4-(4-(2,2,2-trifluoroacetyl)piperazin-1-yl)phenyl)-2-oxooxazolidin-5-yl)methylcarbamothioate;
33. (S)—O-Methyl(3-(4-(4-(2,2-difluorocyclopropanecarbonyl)piperazin-1-yl)-3-fluoro phenyl)-2-oxooxazolidin-5-yl)methylcarbamothioate;
34. (S)—O-Methyl(3-(4-(4-(4-aminonicotinoyl)piperazin-1-yl)-3-fluorophenyl)-2-oxo oxazolidin-5-yl)methylcarbamothioate;
35. (S)—O-Methyl(3-(3-fluoro-4-(4-(6-fluoronicotinoyl)piperazin-1-yl)phenyl)-2-oxooxazolidin-5-yl)methylcarbamothioate;
36. (S)—O-Methyl(3-(4-(4-(2-amino-4-(trifluoromethyl)thiazole-5-carbonyl)piperazin-1-yl)-3-fluorophenyl)-2-oxooxazolidin-5-yl)methylcarbamothioate;
37. (S)—O-Methyl(3-(4-(4-(6-aminonicotinoyl)piperazin-1-yl)-3-fluorophenyl)-2-oxo oxazolidin-5-yl)methylcarbamothioate;
38. (S)—O-Methyl (3-(3-fluoro-4-(4-(5-fluoronicotinoyl)piperazin-1-yl)phenyl)-2-oxo oxazolidin-5-yl)methylcarbamothioate;
39. (S)—O-Methyl(3-(3-fluoro-4-(4-(5-methylisoxazole-3-carbonyl)piperazin-1-yl)phenyl)-2-oxooxazolidin-5-yl)methylcarbamothioate;
40. (S)—O-Methyl(3-(3-fluoro-4-(4-(3-(5-nitrofuran-2-yl)acryloyl)piperazin-1-yl)phenyl)-2-oxooxazolidin-5-yl)methylcarbamothioate;
41. (S)—O-Methyl(3-(4-(4-(2-aminoacetyl)piperazin-1-yl)-3-fluorophenyl)-2-oxo oxazolidin-5-yl)methylcarbamothioate hydrochloride;
42. (S)—O-Methyl(3-(4-(4-((S)-2-amino-3-hydroxypropanoyl)piperazin-1-yl)-3-fluoro phenyl)-2-oxooxazolidin-5-yl)methylcarbamothioate hydrochloride;
43. (S)—O-Methyl(3-(3-fluoro-4-(4-(2-(methylsulfonamido)acetyl)piperazin-1-yl)phenyl)-2-oxooxazolidin-5-ylmethylcarbamothioate;
44. (S)—O-M ethyl(3-(3-fluoro-4-(4-(isoxazole-5-carbonyl)piperazin-1-yl)phenyl)-2-oxooxazolidin-5-yl)methylcarbamothioate;
45. (S)—O-Methyl (3-(4-(4-(2-(benzyloxy)acetyl)piperazin-1-yl)-3-fluorophenyl)-2-oxo oxazolidin-5-yl)methylcarbamothioate;
46. (S)—O-Methyl (3-(4-(4-(cyclohexanecarbonyl)piperazin-1-yl)-3-fluorophenyl)-2-oxooxazolidin-5-yl)methylcarbamothioate;
47. (S)—O-Methyl (3-(4-(4-(cyclopropanecarbonyl)piperazin-1-yl)-3-fluorophenyl)-2-oxooxazolidin-5-yl)methylcarbamothioate;
48. (S)—O-Methyl(3-(4-(4-(cyclopentanecarbonyl)piperazin-1-yl)-3-fluorophenyl)-2-oxooxazolidin-5-yl)methylcarbamothioate;
49. (S)—O-Methyl(3-(4-(4-(2-chloroacetyl)piperazin-1-yl)-3-fluorophenyl)-2-oxooxazolidin-5-yl)methylcarbamothioate;
50. (S)—S-Methyl(3-(4-(4-(4-chlorobenzoyl)piperazin-1-yl)-3-fluorophenyl)-2-oxooxazolidin-5-yl)methylcarbamothioate;
51. (S)—S-Methyl(3-(3-fluoro-4-(4-(morpholine-4-carbonyl)piperazin-1-yl)phenyl)-2-oxooxazolidin-5-yl)methylcarbamothioate;
52. (S)—S-Methyl(3-(3-fluoro-4-(4-(4-fluorobenzoyl)piperazin-1-yl)phenyl)-2-oxo oxazolidin-5-yl)methylcarbamothioate;
53. (S)—S-Methyl(3-(4-(4-(2-chloroacetyl)piperazin-1-yl)-3-fluorophenyl)-2-oxo oxazolidin-5-yl)methylcarbamothioate;
54. (S)—S-Methyl(3-(4-(4-(3-chlorobenzoyl)piperazin-1-yl)-3-fluorophenyl)-2-oxooxazolidin-5-yl)methylcarbamothioate;
55. (S)—S-Methyl (3-(3-fluoro-4-(4-pivaloylpiperazin-1-yl)phenyl)-2-Oxooxazolidin-5-yl)methylcarbamothioate;
56. (S)—S-Methyl(3-(3-fluoro-4-(4-(morpholine-4-carbonothioyl)piperazin-1-yl)phenyl) 2-oxooxazolidin-5-yl)methylcarbamothioate;
57. (S)—S-Methyl(3-(4-(4-(2-(2,5-dichlorophenylthio)acetyl)piperazin-1-yl)-3-fluoro phenyl)-2-oxooxazolidin-5-yl)methylcarbamothioate;
58. (S)—S-Methyl(3-(4-(4-(imidazole-1-carbonyl)piperazin-1-yl)-3-fluorophenyl)-2-oxo oxazolidin-5-yl)methylcarbamothioate;
59. (S)—S-Methyl(3-(3-fluoro-4-(4-(1-methyl-indazole-3-carbonyl)piperazin-1-yl)phenyl)-2-oxooxazolidin-5-yl)methylcarbamothioate;
60. (S)—S-Methyl(3-(4-(4-(2-cyanoacetyl)piperazin-1-yl)-3-fluorophenyl)-2-oxooxazolidin-5-yl)methylcarbamothioate;
61. (S)—S-Methyl(3-(4-(4-(5-chlorothiophene-2-carbonyl)piperazin-1-yl)-3-fluorophenyl)-2-oxooxazolidin-5-yl)methylcarbamothioate;
62. (S)—S-Methyl(3-(4-(4-(2-aminonicotinoyl)piperazin-1-yl)-3-fluorophenyl)-2-oxooxazolidin-5-yl)methylcarbamothioate;
63. (S)—S-Methyl(3-(3-fluoro-4-(4-(6-fluoro-2-naphthoyl)piperazin-1-yl)phenyl)-2-oxooxazolidin-5-yl)methylcarbamothioate;
64. (S)—S-Methyl(3-(4-(4-(2,2-difluorocyclopropanecarbonyl)piperazin-1-yl)-3-fluorophenyl)-2-oxooxazolidin-5-yl)methylcarbamothioate;
65. (S)—S-Methyl(3-(3-fluoro-4-(4-(6-fluoropicolinoyl)piperazin-1-yl)phenyl)-2-oxo oxazolidin-5-yl)methylcarbamothioate;
66. (S)—S-Methyl(3-(3-fluoro-4-(4-(5-methyl isoxazole-3-carbonyl)piperazin-1-yl)phenyl)-2-oxooxazolidin-5-yl)methylcarbamothioate;
67. (S)—S-Methyl(3-(4-(4-(4-aminonicotinoyl)piperazin-1-yl)-3-fluorophenyl)-2-oxooxazolidin-5-yl)methylcarbamothioate;
68. (S)—S-Methyl(3-(4-(4-(3,5-dimethylisoxazole-4-carbonyl)piperazin-1-yl)-3-fluoro phenyl)-2-oxooxazolidin-5-yl)methylcarbamothioate;
69. (S)—S-Methyl (3-(4-(4-(2-amino-4-(trifluoromethyl)thiazole-5-carbonyl)piperazin-1-yl)-3-fluorophenyl)-2-oxooxazolidin-5-yl)methylcarbamothioate;
70. (S)—S-Methyl (3-(3-fluoro-4-(4-(7-fluoro-3-methylbenzofuran-2-carbonyl)piperazin-1-yl)phenyl)-2-oxooxazolidin-5-yl)methylcarbamothioate;
71. (S)—S-Methyl(3-(3-fluoro-4-(4-(3-methylisoxazole-4-carbonyl)piperazin-1-yl)phenyl)-2-oxooxazolidin-5-yl)methylcarbamothioate;
72. (S)—O-Methyl (3-(4-(4-(2-(1H-tetrazol-1-yl)acetyl)piperazin-1-yl)-3-fluorophenyl)-2-oxooxazolidin-5-yl)methylcarbamothioate;
73. (S)—O-M ethyl (3-(3-fluoro-4-(4-(1-hydroxycyclopropanecarbonyl)piperazin-1-yl)phenyl)-2-oxooxazolidin-5-yl)methyl carbamothioate;
74. (S)—O-Methyl(3-(4-(4-(2-(1,2,3-triazol-1-yl)acetyl)piperazin-1-yl)-3-fluorophenyl)-2-oxooxazolidin-5-yl)methylcarbamothioate;
75. (S,E)-O-Methyl(3-(3-fluoro-4-(4-(6-(N′-hydroxycarbamimidoyl)nicotinoyl)piperazin-1-yl)phenyl)-2-oxooxazolidin-5-yl)methylcarbamothioate;
76. (S)—O-Methyl(3-(4-(4-(1H-imidazole-1-carbonothioyl)piperazin-1-yl)-3-fluoro phenyl)-2-oxooxazolidin-5-yl)methylcarbamothioate;
77. (S)—O-Methyl(3-(3-fluoro-4-(4-(2-(2-hydroxyethoxy)acetyl)piperazin-1-yl)phenyl)-2-oxooxazolidin-5-yl)methylcarbamothioate and
78. (S)—O-Methyl(3-(4-(4-(2-(2,3-dihydroxypropoxy)acetyl)piperazin-1-yl)-3-fluoro phenyl)-2-oxooxazolidin-5-yl)methylcarbamothioate.

According to an embodiment of the present invention, a process is provided for the preparation of novel oxazolidinone derivatives of the formula (I) where X and Y represents O or S, and all other symbols are as defined earlier, which comprises (i) Converting the compound of formula (II)

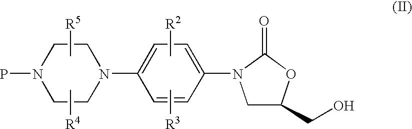

wherein P represents protecting groups such as benzyl, benzyloxy carbonyl, t-butyloxycarbonyl, chloroethyl formate, 9H-(f)luoren-9-yl(m)eth(O)xy(c)arbonyl and all other symbols are as defined earlier to produce a compound of formula (III)

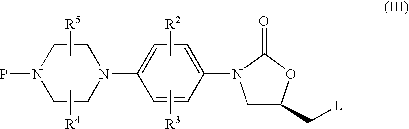

wherein L represents a leaving group such as mesylate, tosylate or triflate and all other symbols are as defined earlier,
ii) Converting the compound of formula (III) to produce a compound of formula (IV)

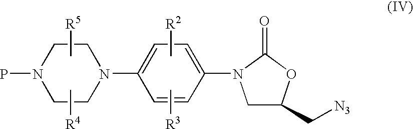

wherein all symbols are as defined earlier,
iii) Reducing the compound of formula (IV) to a compound of formula (V)

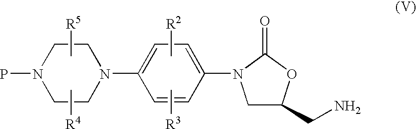

wherein all symbols are as defined earlier,
iv) Converting the compound of formula (V) to produce a compound of formula (VI)

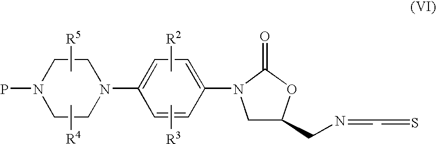

wherein all symbols are as defined earlier,
v) Converting the compound of formula (VI) or by converting compound of formula (V) to produce a compound of formula (VII)

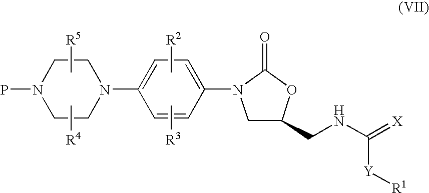

wherein all symbols are as defined earlier and
vi) Deprotecting the compound of formula (VII) to produce a compound formula (VIII),

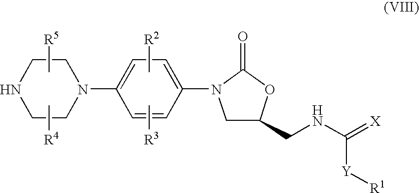

wherein all symbols are as defined earlier,
vii) Reacting the compound of formula (VIII) with a compound of formula (IX)

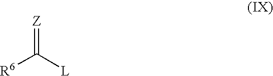

wherein L is a leaving group, preferably z is oxygen and all other symbols are as defined earlier to produce a compound of formula (I).

The compound of formula (II) may be converted to a compound of formula (III) using methane sulfonyl chloride, tosyl chloride, and trifluoromethane sulfonyl chloride. The reaction may be carried out in presence of solvents like tetrahydrofuran, chloroform, dichloromethane, dichloroethane, ethylacetate, o-dichlorobenzene and the like or a mixture thereof and a base selected from dimethylamino pyridine, triethylamine, pyridine and the like. The reaction may be carried out at a temperature in the range of −10° C. to room temperature. The duration of the reaction may range from 1 to 12 hours.

The conversion of compound of formula (III) may be carried out in the presence or one or more equivalents of metal azide such as LiN₃, NaN₃ or trialkyl silylazide. The reaction may be carried out in the presence of a solvent such as THF, acetone, DMF, DMSO and the like or mixtures thereof. The reaction may be carried out in inert atmosphere, which may be maintained using N₂ or Ar. The reaction may be carried out at a temperature in the range of ambient temperature to reflux temperature of the solvent used, preferably at a temperature in the range of 60° C. to 120° C. The reaction time may range from 0.5 to 18 hours.

The reduction of compound of formula (IV) may be carried out in the presence of gaseous, hydrogen and a catalyst such as Ru, Pd, Rh, Pt, Ni on solid beads such as charcoal, alumina, asbestos and the like. The reduction may be conducted in the presence of a solvent such as dioxane, acetic acid, ethyl acetate, THF, alcohols such as methanol, ethanol, isopropanol and the like or mixtures thereof. A pressure between atmospheric pressure to 80 psi may be used. The reaction may be carried out at a temperature in the range of 25 to 60° C., preferably at room temperature. The reaction time ranges from 2 to 48 hours. The reduction may also be carried out by employing metals in mineral acids such as Sn/HCl, Fe/HCl, Zn/HCl, Zn/CH₃CO₂H, Zn—HCO₂H/HCOONH₄ and the like.

The conversion of compound of formula (V) to produce compound of formula (VI) may be carried out using thiophosgene or phosgene gas in the presence of a solvent such as tetrahydrofuran, chloroform, dichloromethane, dichloroethane, ethylacetate, o-dichlorobenzene or a mixture thereof. The reaction may be carried out in the presence of a base selected from dimethylamino pyridine, triethylamine, pyridine and the like. The reaction may be carried out at a temperature in the range of −10° C. to room temperature.

The conversion of compound of formula (VI) to compound of formula (VII) may be carried out using alcohols such as methanol, ethanol, propanol and the like. The reaction may be carried out in the presence of a base selected from dimethylamino pyridine, triethylamine, pyridine and the like. The reaction may be carried out at a temperature in the range of 30° C. to reflux temperature. The duration of the reaction may range from 6 to 18 hours.

The conversion of compound of formula (V) to produce compound of formula (VII) may be carried out using alkylhaloformates such as chloroethylchloroformate, arylchloroformates such as benzylchloroformates, heteroarylchloroformates in presence of organic bases selected from dimethylamino pyridine, triethylamine, pyridine and the like or in presence of inorganic bases selected from sodium bicarbonate, sodium carbonate and cesium carbonate and the like, in presence of solvents such as dichloromethane, acetone, DMF, water, THF and the like or mixtures thereof. The reaction temperature may range from −20° C. to room temperature. The duration of the reaction may range from 3 to 18 hours.

The deprotection of compound of formula (VII) may be carried out using an inorganic acid such as trifluoroacetic acid, hydrochloric acid and sulfuric acid. The reaction may be carried out in presence of appropriate solvents like tetrahydrofuran, chloroform, dichloromethane, dichloroethane, ethylacetate, o-dichlorobenzene or a mixture thereof. The reaction may be carried out at a temperature in the range of 0° C. to 40° C. The duration of the reaction may range from 1 to 6 hours.

The reaction of compound of formula (VIII) with compound of formula (IX) may be carried out in solvents such as toluene, dimethylformamide, tetrahydrofuran, chloroform, dichloromethane, dichloroethane, dioxane, ethylacetate, o-dichlorobenzene or a mixture thereof with or without organic or inorganic bases such as triethyl amine, pyridine, dimethylaminopyridine, sodium carbonate, cesium carbonate, sodium bicarbonate, potassium carbonate, potassium bicarbonate, sodium hydroxide, potassium hydroxide and the like in the absence or presence of reagents such as (DCC) dicyclohexylcarbodiimide, 1-ethyl-3-(3-dimethylaminopropyl)carbodiimide (EDAC), benzotriazole-1-yl-oxy-tris-(dimethylamino)-phosphonium hexafluorophosphate (BOP), N-hydroxysuccinimide (NHS), 1-hydroxybenztriazole hydrate (HOBt) and the like. The reaction may be carried out at a temperature in the range of 0° C. to 40° C. The duration of the reaction may range from 1 to 2 hours.

In yet another embodiment of the present invention, there is provided a process for the preparation of compounds of formula where all the symbols are as defined earlier which comprises reacting the compound of formula (X)

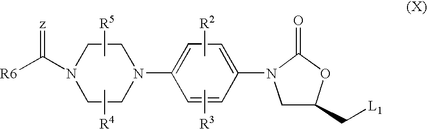

where L₁ represents a leaving group such as mesylate, tosylate or triflate with azides to produce a compound of formula (XI)
i) Converting the compound of formula (X) to produce a compound of formula (XI)

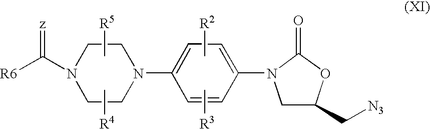

wherein all symbols are as defined earlier,

The conversion of compound of formula (X) may be carried out in the presence of one or more equivalents of metal azide such as LiN₃, NaN₃ or trialkyl silylazide. The reaction may be carried out in the presence of a solvent such as THF, acetone, DMF, DMSO and the like or mixtures thereof. The reaction may be carried out in an inert atmosphere, which may be maintained using N₂ or Ar. The reaction may be carried out at a temperature in the range of ambient temperature to reflux temperature of the solvent used, preferably at a temperature in the range of 60° C. to 120° C. The reaction time may range from 0.5 to 18 hours.

ii) Reducing the compound of formula (XI) to a compound of formula (XII),

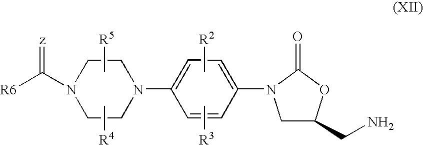

wherein all symbols are as defined earlier.
iii) Converting the compound of formula (XII) to produce a compound of formula (XIII),

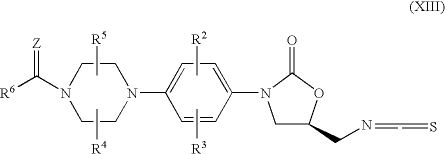

wherein all symbols are as defined earlier.
iv) Converting the compound of formula (XIII) or by alternatively converting the compound of formula (XII) to produce a compound of formula (I),

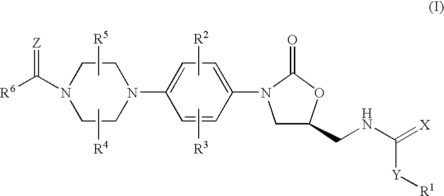

where all symbols are as defined earlier.

In another embodiment of the present invention, a process is provided for the conversion of compounds of formula (I) where X or Z represents the O and all other symbols are as defined above to compounds of formula (I) where X or Z is S, and all other symbols are as defined earlier. The conversion may be carried out in presence of acid or using Lawesson's reagent in the presence of base such as triethyl amine, pyridine and the like and solvents such as toluene, DCC, tetrahydrofuran, chloroform, dichloromethane, dichloroethane, ethylacetate, o-dichlorobenzene or a mixture thereof. The reaction may be carried out at a temperature in the range of 20° C. to 120° C. The duration of the reaction may range from 1 to 12 hours.

According to another embodiment of the present invention, there is provided a process for the preparation of compounds of formula (II),

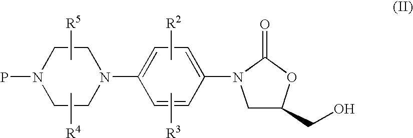

wherein all symbols are as defined earlier, which comprises:
i) Reacting the compound of formula (IIa),

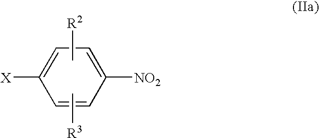

wherein X represents halogen atom and all other symbols are as defined earlier, with compound of formula (IIb),

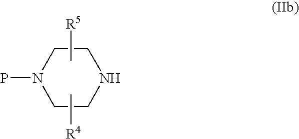

wherein P represents protecting group and all other symbols are as defined earlier, to produce compound of formula (IIe).

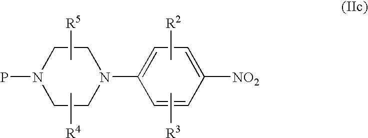

ii) Reducing the compound of formula (IIc) to produce a compound of formula (IId),

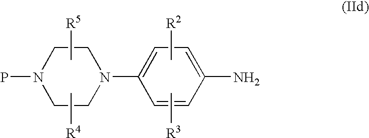

wherein all symbols are as defined earlier.
iii) Converting the compound of formula (IId) to produce compound of formula (IIe),

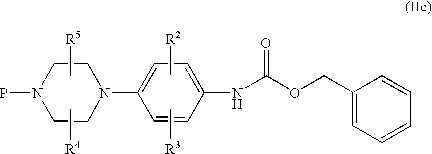

wherein all symbols are as defined earlier.
iv) Cyclizing the compound of formula (IIe) with R-(−)-glycidyl butyrate to produce a compound of formula (II) where all symbols are as defined earlier.

The reaction of compound of formula (IIa) with the compound of formula (IIb) may be carried out in the presence of BINAP [(R)-2,2′-Bis(diphenylphosphino)-1,1′-binaphthyl] and tris(dibenzylidene acetone)dipalladium(0). The reaction may be carried out using inert gases such as N₂, argon and the like. The reaction may be carried out in the presence of solvents such as toluene, DCC, tetrahydrofuran, chloroform, dichloromethane, dichloroethane, ethylacetate, o-dichlorobenzene or a mixture thereof. The reaction is carried out at temperature in the range of 20° C. to 60° C.

The reduction of compound of formula (IIc) may be carried out in the presence of gaseous hydrogen and a catalyst such as Ru, Pd, Rh, Pt, Ni on solid beads such as charcoal, alumina, asbestos and the like. The reduction may be conducted in the presence of a solvent such as dioxane, acetic acid, ethyl acetate, THF, alcohol such as methanol, ethanol, isopropanol and the like or mixtures thereof. A pressure between atmospheric pressure to 80 psi may be used. The reaction may be carried out at a temperature in the range of 25 to 60° C., preferably between 30° C. and 40° C. The reaction time ranges from 2 to 48 hours. The reduction may also be carried out by employing metals in mineral acids such as Sn/HCl, Fe/HCl, Zn/HCl, Zn/CH₃CO₂H, Zn/HCO₂H/HCOONH₄ and the like.

The conversion of compound of formula (IId) to compound of formula (IIe) may be carried out using benzyloxycarbonyl chloride in the presence of inorganic or organic base such as sodium bicarbonate, caesium carbonate, potassium carbonate, triethylamine, diisopropylamine, N,N-dimethylaniline, lutidine and the like, in solvents such as acetone, DMF, water, THF and the like or mixtures thereof. The reaction temperature may range from −20° C. to 40° C. The duration of the reaction may range from 2 to 18 hours.

The cyclization of compound of formula (IIe) may be carried out in the presence of base such as n-butyl lithium, LDA, potassium bis(trimethylsilyl)amide, lithium-bis(trimethylsilyl)amide and the like. The reaction may be carried out in the presence of solvent such as THF, DMF and the like. The reaction is carried out using clhiral ester such as R-(−)-glycidyl butyrate. The reaction is carried out at a temperature in the range of −80° C. to −50° C. The duration of the reaction may range from 2 to 12 hours.

It is appreciated that in any of the above-mentioned reactions, any reactive group in the substrate molecule may be protected according to conventional chemical practice. Suitable protecting groups in any of the above-mentioned reactions are those used conventionally in the art. The methods of formation and removal of such protecting groups are those conventional methods appropriate to the molecule being protected.

The pharmaceutically acceptable salts are prepared by reacting the compound of formula (I) with 1 to 4 equivalents of a base such as sodium hydroxide, sodium methoxide, sodium hydride, potassium t-butoxide, calcium hydroxide, magnesium hydroxide and the like, in solvents like ether, tetrahydrofuran, methanol, t-butanol, dioxane, isopropanol, ethanol etc. Mixture of solvents may be used. Organic bases such as diethanolamine, α-phenylethylamine, benzylamine, piperidine, morpholine, pyridine, hydroxyethylpyrrolidine, hydroxyethylpiperidine, choline and the like, ammonium or substituted ammonium salts, aluminum salts. Amino acid such as glycine, alanine, cystine, cysteine, lysine, arginine, phenylalanine, guanidine etc may be used for the preparation of amino acid salts. Alternatively, acid addition salts wherever applicable are prepared by the treatment with acids such as hydrochloric acid, hydrobromic acid, nitric acid, sulfuric acid, phosphoric acid, p-toluenesulphonic acid, methanesulfonic acid, acetic acid, citric acid, maleic acid, salicylic acid, hydroxynaphthoic acid, ascorbic acid, palmitic acid, succinic acid, benzoic acid, benzenesulfonic acid, tartaric acid and the like in solvents like ethyl acetate, ether, alcohols, acetone, tetrahydrofuran, dioxane etc. Mixture of solvents may also be used.

The stereoisomers of the compounds forming part of this invention may be prepared by using reactants in their single enantiomeric form in the process wherever possible or by conducting the reaction in the presence of reagents or catalysts in their single enantiomer form or by resolving the mixture of stereoisomers by conventional methods. Some of the preferred methods include use of microbial resolution, resolving the diastereomeric salts formed with chiral acids such as mandelic acid, camphorsulfonic acid, tartaric acid, lactic acid, and the like wherever applicable or chiral bases such as brucine, cinchona alkaloids and their derivatives and the like.

More specifically the compound of formula (I) may be converted to a 1:1 mixture of diastereomeric amides by treatment with chiral amines, aminoacids, aminoalcohols derived from aminoacids; conventional reaction conditions may be employed to convert the acid into an amide; the diastereomers may be separated either by fractional crystallization or chromatography and the stereoisomers of compound of formula (I) may be prepared by hydrolysing the pure diastereomeric amide.

Various polymorphs of compound of general formula (I) forming part of this invention may be prepared by crystallization of compound of formula (I), under different conditions. For example, using different solvents commonly used or their mixtures for recrystallization; crystallizations at different temperatures; various modes of cooling, ranging from very fast to very slow cooling during crystallizations. Polymorphs may also be obtained by gradual or fast cooling of compound after heating or melting. The presence of polymorphs may be determined by solid probe NMR spectroscopy, IR spectroscopy, differential scanning calorimetry, powder X-ray diffraction or such other techniques.

Pharmaceutically acceptable solvates of the compounds of formula (I) forming part of this invention may be prepared by conventional methods such as dissolving the compounds of formula (I) in solvents such as water, methanol, ethanol, mixture of solvents such as acetone-water, dioxane-water, N,N-dimethylformamide-water and the like, preferably water and recrystallizing by using different crystallization techniques.

It should be noted that compounds of the invention may contain groups that may exist in tautomeric forms, and though one form is named, described, displayed and/or claimed herein, all the forms are intended to be inherently included in such name, description, display and/or claim.

Prodrugs of the compounds of formula (I), are also contemplated by this invention. A prodrug is an active or inactive compound that is modified chemically through in-vivo physiological action, such as hydrolysis, metabolism and the like, into a compound of this invention following administration of the prodrug to a patient. The suitability and techniques involved in making and using prodrugs are well known to those skilled in the art.

The present invention also provides a pharmaceutical composition, containing one or more of the compounds of the general formula (I), as defined above, their derivatives, analogs, tautomeric forms, stereoisomers, polymorphs, hydrates, metabolites, prodrugs, pharmaceutically acceptable salts, pharmaceutically acceptable solvates in combination with the usual pharmaceutically employed carriers, diluents and the like.

The derivatives provided by the present invention can be employed as pharmaceutical compositions, for example, in the form of pharmaceutical compositions containing the derivatives together with appropriate, pharmaceutically acceptable carriers. The products in accordance with the invention can be administered, for example, perorally, such as in the form of tablets, coated tablets, dragees, hard and soft gelatine capsules, solutions, emulsions or suspensions, or rectally, such as in the form of suppositories, etc. The compositions may be sterilized and may contain auxiliary agents generally employed in the pharmaceutical art, such as sodium hydrogen carbonate, citric acid, propylene glycol, tween 80, etc. The compounds can be used orally or parenterally.

The pharmaceutical composition may be in the forms normally employed, such as tablets, capsules, powders, syrups, solutions, suspensions and the like, may contain flavorants, sweeteners etc. in suitable solid or liquid carriers or diluents, or in suitable sterile media to form injectable solutions or suspensions. The compositions may be prepared by processes known in the art, such as by combining the ingredients into a dosage form together with suitable, non-toxic, inert, therapeutically compatible solid or liquid carrier materials and if desired, the usual pharmaceutical adjuvants. The amount of the active ingredient in the composition may be less than 70% by weight. Such compositions typically contain from 1 to 25%, preferably 1 to 15% by weight of active compound, the remainder of the composition being pharmaceutically acceptable carriers, diluents, excipients or solvents.

Suitable pharmaceutically acceptable carriers include solid fillers or diluents and sterile aqueous or organic solutions. The active compound will be present in such pharmaceutical compositions in the amounts sufficient to provide the desired dosage in the range as described above. Thus, for oral administration, the compounds can be combined with a suitable solid or liquid carrier or diluent to form capsules, tablets, powders, syrups, solutions, suspensions and the like. The pharmaceutical compositions, may, if desired, contain additional components such as flavorants, sweeteners, excipients and the like. For parenteral administration, the compounds can be combined with sterile aqueous or organic media to form injectable solutions or suspensions. For example, solutions in sesame or peanut oil, aqueous propylene glycol and the like can be used, as well as aqueous solutions of water-soluble pharmaceutically-acceptable acid addition salts or alkali or alkaline earth metal salts of the compounds. The injectable solutions prepared in this manner can then be, administered intravenously, intraperitoneally, subcutaneously, or intramuscularly, with intramuscular administration being preferred in humans.

Generally, the effective dose for treating a particular condition in a patient may be readily determined and adjusted by the physician during treatment to alleviate the symptoms or indications of the condition or disease. Generally, a daily dose of active compound in the range of about 0.01 to 1000 mg/kg of body weight is appropriate for administration to obtain effective results. The daily dose may be administered in a single dose or divided into several doses. In some cases, depending upon the individual response, it may be necessary to deviate upwards or downwards from the initially prescribed daily dose. Typical pharmaceutical preparations normally contain from about 0.2 to about 500 mg of active compound of formula I and/or its pharmaceutically active salts or solvates per dose.

While the compounds of the invention can be administered as the sole active pharmaceutical agent, they can also be used in combination with one or more compounds of the invention or other agents. When administered as a combination, the therapeutic agents can be formulated as separate compositions that are given at the same time or different times, or the therapeutic agents can be given as a single composition.

The term “therapeutically effective amount” or “effective amount” refers to that amount of a compound or mixture of compounds of Formula I that is sufficient to effect treatment, as defined below, when administered alone or in combination with other therapies to a mammal in need of such treatment.

The term “animal” as used herein is meant to include all mammals, and in particular humans. Such animals are also referred to herein as subjects or patients in need of treatment. The therapeutically effective amount will vary depending upon the subject and disease condition being treated, the weight and age of the subject, the severity of the disease condition, the particular compound of Formula I chosen, the dosing regimen to be followed, timing of administration, the manner of administration and the like, all of which can readily be determined by one of ordinary skill in the art.

The term “treatment” or “treating” means any treatment of a disease in a mammal, including:

a) Preventing the disease, that is, causing the clinical symptoms of the disease not to develop;

b) Inhibiting the disease, that is, slowing or arresting the development of clinical symptoms; and/or

c) Relieving the disease, that is, causing the regression of clinical symptoms.

From the foregoing description, one skilled in the art can easily ascertain the essential characteristics of this invention, and without departing from the spirit and scope thereof, make various changes and modifications of the invention to adapt it to various usages and conditions.

Finally, the compounds of the present invention are useful for the treatment of microbial infections in humans and other warm-blooded animals, under both parenteral and oral administration. In addition to the compounds of formula (I) the pharmaceutical compositions of the present invention may also contain or be co-administered with one or more known drugs selected from other clinically useful antibacterial agents such as β-lactams or aminoglycosides. These may include penicillins such as oxacillin or flucloxacillin and carbapenems such as meropenem or imipenem to broaden the therapeutic effectiveness against, for example methicillin-resistant staphylococci. Compounds of the formula (I), may also contain or be co-administered with bactericidal/permeability-increasing-G protein product (BPI) or efflux pump inhibitors to improve activity against gram negative bacteria and bacteria resistant to anti-microbial agents.

The present invention is provided by the examples given below, which are provided by the way of illustration only, and should not be considered to limit the scope of the invention. Variation and changes, which are obvious to one skilled in the art, are intended to be within the scope and nature of the invention, which are defined in the appended claims.

### Example 1

### Synthesis of (S)—O-methyl (3-(4-(4-(benzofuran-2-carbonyl)piperazin-1-yl)-3-fluorophenyl)-2-oxooxazolidin-5-yl)methylearbamothioate

### Step 1

### Preparation of (R)-t-butyl 4-(2-fluoro-4-(5-(isothioeyanatomethyl)-2-oxooxazolidin-3-yl)phenyl)piperazine-1-carboxylate

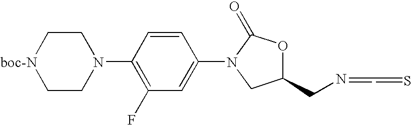

To a cold stirred solution (0° C.) of (S)-t-butyl 4-(4-(5-(aminomethyl)-2-oxo oxazolidin-3-yl)-2-fluorophenyl)piperazine-1-carboxylate (10.0 g, 25.3 mmol, prepared according to the procedure described in EPI 130016) in dry DCM (100 ml) was added triethyl amine (7.03 ml, 50.6 mmol). To the resulted reaction mass, was added thiophosgene (2.3 ml, 30.4 mmol) at the same temperature. The stirring was continued for 3 hours allowing it to come to room temperature, subsequently, the solvent was removed from the reaction mixture over rotary evaporator and the residue was dissolved in ethyl acetate (500 ml). The ethyl acetate layer was washed with sodium bicarbonate, water and brine solution, dried over anhydrous Na₂SO₄ and concentrated under high vacuum, to afford the title compound (11.1 g, 99.5%) that was used directly in the next step without further purification. Mass (m/z): 437.1 (M⁺+1).

### Step 2

### Synthesis of (S)-1-butyl 4-(2-fluoro-4-(5-((methoxycarbonothioylamino) methyl)-2-oxooxazolidin-3-yl)phenyl)piperazine-1-carboxylate

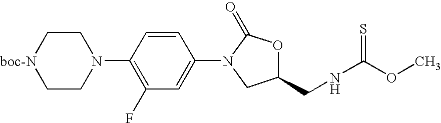

A solution of (R)-t-butyl 4-(2-fluoro-4-(5-(isothiocyanatomethyl)-2-oxooxazolidin-3-yl)phenyl)piperazine-1-carboxylate, obtained according to the procedure described in step-1, (8.0 g, 1.91 mmol) in methanol (80 ml) was heated to reflux temperature for 6 hours. After completion of the reaction, the solvent was removed and the reaction mass was subjected to column chromatography over silica gel using hexane and ethyl acetate mixture as an eluent to yield the title compound (5.2 g, 60%). ¹H-NMR (DMSO-d₆): δ 2.50 (s, 9H), 2.90-2.92 (m, 4H), 3.43-3.47 (m, 5H), 3.75-3.79 (m, 2H), 3.83-3.93 (2 s (rotational isomers), 3H), 4.12 (m, 1H), 4.86-4.88 (2 m, (rotational isomers), 1H), 7.06-7.10 (m, 1H), 7.16-7.19 (m, 1H), 7.45-7.51 (m, 1H), 9.44-9.53(2 m, (rotational isomers), 1H, D₂O exchangeable). Mass (m/z): 469.1 (M⁺+1).

### Step 3

### Synthesis of (S)—O-methyl(3-(3-fluoro-4-(piperazin-1-yl)phenyl)-2-oxooxazolidin-5-yl)methylcarbamothioate

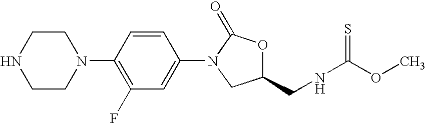

Trifluoroacetic acid (16.4 ml, 0.2 mmol) was added to a cold (0° C.) solution of (S)-t-butyl-4-(2-fluoro-4-(5-((methoxycarbonothioylamino)methyl)-2-oxooxazolidin-3-yl)phenyl)piperazine-1-carboxylate (5.0 g, 10.6 mmol), obtained according to the procedure given in the step 2, in dry DCM (100 ml) under stirring. After completion of the addition the reaction mixture was allowed to stir at room temperature for 3 hours. To this resulted reaction mass, was added excess of saturated sodium bicarbonate solution and the stirring was continued for another 15 minutes. The solid was then filtered and washed thoroughly with ethyl acetate. The filtrate was concentrated to dryness and dried under vacuum to afford the title compound (3.9 g, 99%) that was used directly in the next step without further purification.

### Step 4

### Synthesis of (S)—O-methyl (3-(4-(4-(benzofuran-2-carbonyl)piperazin-1-yl)-3-fluorophenyl)-2-oxooxazolidin-5-yl)methylcarbamothioate

To a solution of (S)—O-methyl (3-(3-fluoro-4-(piperazin-1-yl)phenyl)-2-oxo-oxazolidin-5-yl)methylcarbamothioate (0.49 g, 1.3 mmol) (prepared according to the procedure described in the step 3) in DMF (7 ml), benzofuran-2-carboxylic acid (0.23 g, 1.5 mmol), 1-hydroxybenzotriazole hydrate (HOBt) (0.18 g, 1.3 mmol), 1-ethyl-3-(3-dimethylaminopropyl) carbodiimide hydrochloride (EDC) (0.51 g, 2.60 mmol) and triethyl amine (0.5 ml, 3.9 mmol) were added under stirring and the resulted reaction mass was stirred for 15 hours. The reaction mass was then poured in to the water and the organic parts were extracted with EtOAc; the ethyl acetate layer was dried over anhydrous Na₂SO₄ and evaporated under vacuum. The crude product was purified by flash chromatography on silica gel using a Biotage SP-1 system (12+M) cartridge using ethyl acetate and hexane as eluent to afford the title compound (0.12 g, yield 17.2%).

¹H-NMR (DMSO-d₆): δ 3.07-3.08 (m, 4H), 3.75-3.82 (m, 3H), 3.88-3.93 (m, 7H), 4.10-4.15 (m, 1H), 4.89 (2 m, (rotational isomers), 1H), 7.12-7.14 (m, 1H), 7.19-7.21 (m, 1H), 7.35-7.37 (m, 1H), 7.44-7.53 (m, 3H), 7.67-7.77 (m, 2H), 9.53-9.55 (2 m, (rotational isomers), 1H, D₂O exchangeable). Mass (m/z): 513.1 (M⁺+1).

The following compounds were prepared from the intermediate obtained in step 11 followed by the procedure described in step 12 of Example 1.

| Ex No | IUPAC name | Analytical Data |
|---|---|---|
| 2 | (S)—O-Methyl (3-(3-fluoro-4-(4- | ¹H-NMR (DMSO-d₆): δ |
| | nicotinoylpiperazin-1-yl)phenyl)-2-oxo | 2.50-2.98 (m, 4H), 3.49-4.12 (m, |
| | oxazolidin-5-yl)methyl carbamothioate | 11H), 4.87-4.88 (m, 1H), |
| | | 7.07-8.67 (m, 7H), 9.53-9.55 (2m, |
| | | (rotational isomers), 1H, D₂O |
| | | exchangeable). Mass (m/z): |
| | | 474.1 (M⁺ + 1) |
| 3. | (S)—O-Methyl (3-(3-fluoro-4-(4- | ¹H-NMR (DMSO-d₆): δ |
| | (pyrazine-2-carbonyl)piperazin-1-yl)phenyl)- | 2.98-3.08 (m, 4H), 3.59-4.12 (m, |
| | 2-oxooxazolidin-5-yl)methyl | 11H), 4.87-4.88 (2m, (rotational |
| | carbamothioate | isomers), 1H), 7.10-8.89 (m, |
| | | 6H), 9.52-9.54 (2m, (rotational |
| | | isomers), 1H, D₂O exchangeable). |
| | | Mass (m/z): 475.1 (M⁺ + 1). |
| 4. | (S)—O-Methyl (3-(3-fluoro-4-(4-(3,4,5- | ¹H-NMR (DMSO-d₆): δ |
| | trimethoxybenzoyl)piperazin-1-yl)phenyl)- | 0.82-0.86 (m, 2H), 3.0-4.12 (m, 22H), |
| | 2-oxooxazolidin-5-yl)methyl | 4.87-4.88 (2m, (rotational isomers), |
| | carbamothioate | 1H), 6.72-7.52 (m, 5H), |
| | | 9.51-9.54 (2m, (rotational isomers), 1H, |
| | | D₂O exchangeable). Mass (m/z): |
| | | 563.2 (M⁺ + 1). |
| 5. | (S)—O-Methyl (3-(4-(4-(4-acetamido | ¹H-NMR (DMSO-d₆): δ 2.06 (s, |
| | benzoyl)piperazin-1-yl)-3-fluorophenyl)- | 3H), 2.99 (brs, 4H), 3.6-4.12 (m, |
| | 2-oxooxazolidin-5-yl)methyl | 11H), 4.87-4.88 (2m, (rotational |
| | carbamothioate | isomers), 1H), 7.07-7.66 (m, |
| | | 7H), 9.52-9.54 (2m, (rotational |
| | | isomers), 1H, D₂O exchangeable), |
| | | 10.13 (s, 1H, D₂O exchangeable). |
| | | Mass (m/z): 530.2 (M⁺ + 1). |
| 6 | (S)—O-Methyl(3-(3-fluoro-4-(4-(5- | ¹H-NMR (DMSO-d₆): δ 2.56 (s, |
| | methylpyrazine-2-carbonyl) piperazin- | 3H), 2.97-3.07 (m, 4H), |
| | 1-yl)phenyl)-2-oxooxazolidin-5-yl)methylcarbamothioate | 3.62-3.82 (m, 7H), 3.88-3.92 (2s |
| | | (rotational isomers), 3H), |
| | | 4.09-4.14 (m, 1H), 4.86-4.88 (2m, |
| | | (rotational isomers), 1H), |
| | | 7.08-7.10 (m, 1H), 7.17-7.20 (m, 1H), |
| | | 7.48-7.51 (m, 1H), 8.58 (s, 1H), |
| | | 8.75 (s, 1H), 9.43-9.54 (2m, |
| | | (rotational isomers), 1H, D₂O |
| | | exchangeable). Mass (m/z): |
| | | 489.1 (M⁺ + 1). |
| 7 | (S)—O-Methyl(3-(3-fluoro-4-(4-(5- | ¹H-NMR (DMSO-d₆): δ 3.05 (m, |
| | nitrofuran-2-carbonyl)piperazin-1-yl)phenyl)- | 4H), 3.69-3.80 (m, 4H), 3.82 (m, |
| | 2-oxooxazolidin-5-yl) methyl | 3H), 3.88-3.93 ((2s (rotational |
| | carbamothioate; | isomers), 3H), 4.10-4.15 (m, 1H), |
| | | 4.89-4.90 (2m, (rotational |
| | | isomers), 1H), 7.09-7.14 (m, 1H), |
| | | 7.18-7.21 (m, 1H), 7.29-7.30 (d, |
| | | 1H), 7.49-7.53 (m, 1H), |
| | | 7.74-7.75 (d, 1H), 9.44-9.55 (2m, (rotational |
| | | isomers), 1H, D₂O exchangeable). |
| | | Mass (m/z): 508.1 (M⁺ + 1). |
| 8 | (S)—O-Methyl(3-(4-(4-(6-cyano | ¹H-NMR (DMSO-d₆): δ |
| | nicotinoyl)piperazin-1-yl)-3-fluorophenyl)- | 2.97-3.08 (m, 4H), 3.46 (m, 2H), |
| | 2-oxooxazolidin-5-yl)methyl | 3.75-3.82 (m, 5H), 3.88-3.93 ((2s |
| | carbamothioate | (rotational isomers), 3H), |
| | | 4.10-4.12 (m, 1H), 4.88-4.90 (2m, |
| | | (rotational isomers), 1H), |
| | | 7.09-7.12 (m, 1H), 7.18-7.19 (m, 1H), |
| | | 7.48-7.52 (m, 1H), 8.16 (m, 2H), |
| | | 8.84 (m, 1H), 9.53 (2m, (rotational |
| | | isomers), 1H, D₂O exchangeable). |
| | | Mass (m/z): 499.1 (M⁺ + 1). |
| 9 | (S)—O-Methyl (3-(3-fluoro-4-(4-(1- | ¹H-NMR (DMSO-d₆): δ |
| | methyl-pyrrole-2-carbonyl)piperazin-1- | 2.99-3.01 (m, 4H), 3.68 (s, 3H), |
| | yl)phenyl)-2-oxooxazolidin-5-yl)methylcarbamothioate | 3.74-3.81 (m, 7H), 3.88-3.93 ((2s |
| | | (rotational isomers), 3H), |
| | | 4.10-4.15 (m, 1H), 4.88-4.89 (2m, |
| | | (rotational isomers), 1H), |
| | | 6.04-6.05 (m, 1H), 6.35-6.36 (m, 1H), |
| | | 6.91 (m, 1H), 7.08-7.13 (m, 1H), |
| | | 7.17-7.20 (m, 1H), 7.48-7.52 (m, |
| | | 1H), 9.52-9.54 (2m, (rotational |
| | | isomers), 1H, D₂O exchangeable). |
| | | Mass (m/z): 476.1 (M⁺ + 1). |
| 10 | (S)—O-Methyl(3-(3-fluoro-4-(4-iso | ¹H-NMR (DMSO-d₆): δ |
| | nicotinoylpiperazin-1-yl)phenyl)-2- | 2.96-3.06 (m, 4H), 3.40-3.42 (m, 3H), |
| | oxooxazolidin-5-yl)methyl | 3.75-3.79 (m, 4H), 3.88-3.93 ((2s |
| | carbamothioate; | (rotational isomers), 3H), |
| | | 4.10-4.14 (m, 1H), 4.86-4.90 (2m, |
| | | (rotational isomers), 1H), |
| | | 7.07-7.12 (m, 1H), 7.18-7.20 (m, 1H), |
| | | 7.43-7.52 (m, 3H), 8.68-8.70 (m, |
| | | 2H), 9.44-9.55 (2m, (rotational |
| | | isomers), 1H, D₂O exchangeable). |
| | | Mass (m/z): 474.1 (M⁺ + 1). |
| 11 | (S)—O-Methyl (3-(3-fluoro-4-(4- | ¹H-NMR (DMSO-d₆): δ |
| | (quinoline-3-carbonyl)piperazin-1-yl)phenyl)- | 3.02-3.10 (m, 4H), 3.37-3.39 (m, 2H), |
| | 2-oxooxazolidin-5-yl)methylcarbamothioate | 3.60 (m, 2H), 3.75-3.82 (m, 3H), |
| | | 3.88-3.93 ((2s (rotational isomers), |
| | | 3H), 4.10-4.13 (m, 1H), |
| | | 4.88-4.90 (2m, (rotational isomers), 1H), |
| | | 7.11-7.14 (m, 1H), 7.20-7.21 (m, |
| | | 1H), 7.49-7.53 (m, 1H), |
| | | 7.68-7.72 (m, 1H), 7.84-7.88 (m, 1H), |
| | | 8.07-8.10 (m, 2H), 8.52 (m, 1H), |
| | | 8.95-8.96 (m, 1H) 9.53-9.55 (2m, |
| | | (rotational isomers), 1H, D₂O |
| | | exchangeable). Mass (m/z): |
| | | 524.1 (M⁺ + 1). |
| 12 | (S)—O-Methyl(3-(4-(4-(6-chloro | ¹H-NMR (DMSO-d₆): δ |
| | nicotinoyl)piperazin-1-yl)-3-fluorophenyl)- | 2.97-3.06 (m, 4H), 3.49 (m, 3H), |
| | 2-oxooxazolidin-5-yl)methyl | 3.74-3.81 (m, 4H), 3.88-3.93 ((2s |
| | carbamothioate | (rotational isomers), 3H), |
| | | 4.10-4.14 (m, 1H), 4.88 (2m, (rotational |
| | | isomers), 1H), 7.07-7.11 (m, 1H), |
| | | 7.18-7.21 (m, 1H), 7.48-7.52 (m, |
| | | 1H), 7.63-7.65 (m, 1H), |
| | | 7.95-7.98 (m, 1H), 8.52 (m, 1H), |
| | | 9.45-9.54 (2m, (rotational isomers), 1H, |
| | | D₂O exchangeable). Mass (m/z): |
| | | 509.1 (M⁺ + 1). |
| 13 | (S)—O-Methyl(3-(4-(4-(1H-indole-2- | ¹H-NMR (DMSO-d₆): δ 3.06 (m, |
| | carbonyl)piperazin-1-yl)-3-fluorophenyl)- | 4H), 3.49-3.81 (m, 7H), |
| | 2-oxooxazolidin-5-yl) methyl | 3.88-3.93 ((2s (rotational isomers), 3H), |
| | carbamothioate; | 4.01-4.12 (m, 1H), 4.88-4.90 (2m, |
| | | (rotational isomers), 1H), 6.84 (m, |
| | | 1H), 7.05-7.41 (m, 4H), |
| | | 7.44-7.62 (m, 3H), 9.45-9.54 (2m, |
| | | (rotational isomers), 1H, D₂O |
| | | exchangeable), 11.61 (s, 1H, D₂O |
| | | exchangeable). Mass (m/z): |
| | | 512.1 (M⁺ + 1). |
| 14 | (S)—O-Methyl(3-(3-fluoro-4-(4-(2- | ¹H-NMR (DMSO-d₆): δ |
| | fluoronicotinoyl)piperazin-1-yl)phenyl)- | 2.95-3.06 (m, 4H), 3.38-3.41 (m, 2H), |
| | 2-oxooxazolidin-5-yl)methyl | 3.75-3.81 (m, 5H), 3.88-3.93 ((2s |
| | carbamothioate | (rotational isomers), 3H), |
| | | 4.10-4.14 (m, 1H), 4.88-4.90 (2m, |
| | | (rotational isomers), 1H), |
| | | 7.08-7.12 (m, 1H), 7.18-7.21 (m, 1H), |
| | | 7.48-7.52 (m, 2H), 8.05-8.10 (m, |
| | | 1H), 8.35-8.36 (m, 1H), |
| | | 9.45-9.54 (2m, (rotational isomers), 1H, D₂O |
| | | exchangeable). Mass (m/z): |
| | | 492.1 (M⁺ + 1). |
| 15 | (S)—O-Methyl(3-(3-fluoro-4-(4-(5- | ¹H-NMR (DMSO-d₆): δ 3.06 (m, |
| | fluoro-1H-indole-2-carbonyl)piperazin- | 4H), 3.40 (m, 2H), 3.76-3.82 (m, |
| | 1-yl)phenyl)-2-oxooxazolidin-5-yl)methylcarbamothioate | 5H), 3.88-3.92 ((2s (rotational |
| | | isomers), 3H), 4.11-4.13 (m, 1H), |
| | | 4.88 (2m, (rotational isomers), 1H), |
| | | 6.83 (s, 1H), 7.05-7.20 (m, 3H), |
| | | 7.35-7.53 (m, 3H), 9.54 (2m, |
| | | (rotational isomers), 1H, D₂O |
| | | exchangeable), 11.74 (1H, s, D₂O |
| | | exchangeable). Mass (m/z): |
| | | 528.1 (M⁺ − 1). |
| 16 | (S)—O-Methyl(3-(4-(4-(4-chloro | ¹H-NMR (DMSO-d₆): δ |
| | nicotinoyl)piperazin-1-yl)-3-fluorophenyl)- | 2.94-3.09 (m, 4H); 3.42 (m, 2H), |
| | 2-oxooxazolidin-5-yl)methylcarbamothioate | 3.75-3.84 (m, 5H), 3.88-3.93 ((2s |
| | | (rotational isomers), 3H), |
| | | 4.10-4.12 (m, 1H), 4.88 (2m, (rotational |
| | | isomers), 1H), 7.08-7.12 (m, 1H), |
| | | 7.18-7.20 (m, 1H), 7.48-7.52 (m, |
| | | 1H), 7.68-7.69 (m, 1H), |
| | | 8.61-8.63 (m, 2H), 9.52-9.55 (2m, |
| | | (rotational isomers), 1H, D₂O |
| | | exchangeable). Mass (m/z): |
| | | 508.1 (M⁺ + 1). |
| 17 | (S)—O-Methyl(3-(3-fluoro-4-(4-(6- | ¹H-NMR (DMSO-d₆): δ |
| | methylnicotinoyl)piperazin-1-yl)phenyl)- | 2.53-2.55 (s, 3H), 2.99-3.03 (m, 4H), |
| | 2-oxooxazolidin-5-yl) methyl | 3.49-3.50 (m, 2H), 3.74-3.81 (m, |
| | carbamothioate | 5H), 3.88-3.93 ((2s (rotational |
| | | isomers), 3H), 4.10-4.14 (m, 1H), |
| | | 4.86-4.89 (2m, (rotational isomers), |
| | | 1H), 7.07-7.12 (m, 1H), |
| | | 7.18-7.20 (m, 1H), 7.34-7.36 (m, 1H), |
| | | 7.48-7.52 (m, 1H), 7.75-7.77 (m, |
| | | 1H), 8.52 (s, 1H), 9.44-9.55 (2m, |
| | | (rotational isomers), 1H, D₂O |
| | | exchangeable). Mass (m/z): |
| | | 489.1 (M⁺ + 1). |
| 18 | (S)—O-Methyl(3-(4-(4-(1H-imidazole-4- | ¹H-NMR (DMSO-d₆): δ 2.99 (m, |
| | carbonyl)piperazin-1-yl)-3-fluorophenyl)- | 4H), 3.76-3.82 (m, 7H), |
| | 2-oxooxazolidin-5-yl) methyl | 3.88-3.93 ((2s (rotational isomers), 3H), |
| | carbamothioate | 4.10-4.14 (m, 1H), 4.86-4.89 (2m, |
| | | (rotational isomers), 1H), |
| | | 7.08-7.19 (m, 2H), 7.48-7.52 (m, 1H), |
| | | 7.65 (s, 1H), 7.73 (s, 1H), |
| | | 9.44-9.53 (2m, (rotational isomers), 1H, |
| | | D₂O exchangeable), 12.50 (s, 1H, |
| | | D₂O exchangeable). Mass (m/z): |
| | | 463.1 (M⁺ + 1). |
| 19 | (S)—O-Methyl(3-(4-(4-(1-cyano | ¹H-NMR (DMSO-d₆): δ |
| | cyclopropanecarbonyl)piperazin-1-yl)- | 1.52-1.64 (m, 4H), 3.04 (m, 4H), |
| | 3-fluorophenyl)-2-oxooxazolidin-5- | 3.75-3.82 (m, 7H), 3.88-3.93 ((2s |
| | yl)methylcarbamothioate | (rotational isomers), 3H), |
| | | 4.10-4.12 (m, 1H), 4.88-4.90 (2m, |
| | | (rotational isomers), 1H), |
| | | 7.08-7.13 (m, 1H), 7.19-7.21 (m, 1H), |
| | | 7.49-7.53 (m, 1H), 9.44-9.55 (2m, |
| | | (rotational isomers), 1H, D₂O |
| | | exchangeable). Mass (m/z): |
| | | 462.1 (M⁺ + 1). |
| 20 | (S)—O-Methyl(3-(4-(4-(5-bromo | ¹H-NMR (DMSO-d₆): δ |
| | nicotinoyl)piperazin-1-yl)-3-fluorophenyl)- | 2.98-3.06 (m, 4H), 3.49 (m, 2H), |
| | 2-oxooxazolidin-5-yl)methylcarbamothioate | 3.76-3.82 (m, 5H), 3.88-3.93 ((2s |
| | | (rotational isomers), 3H), |
| | | 4.10-4.14 (m, 1H), 4.86-4.90 (2m, |
| | | (rotational isomers), 1H), |
| | | 7.07-7.12 (m, 1H), 7.18-7.20 (m, 1H), |
| | | 7.48-7.51 (m, 1H), 8.19 (s, 1H), |
| | | 8.65 (s, 1H), 8.81 (s, 1H), |
| | | 9.45-9.54 (2m, (rotational isomers), 1H |
| | | D₂O exchangeable). Mass (m/z): |
| | | 552 (M⁺). |
| 21 | (S)—O-Methyl (3-(3-fluoro-4-(4- | ¹H-NMR (DMSO-d₆): δ |
| | (quinoxaline-2-carbonyl)piperazin-1- | 3.03-3.13 (m, 4H), 3.49 (m, 2H), |
| | yl)phenyl)-2-oxooxazolidin-5-yl)methylcarbamothioate | 3.74-3.82 (m, 5H), 3.88-3.92 ((2s |
| | | (rotational isomers), 3H), |
| | | 4.10-4.15 (m, 1H), 4.88 (2m, (rotational |
| | | isomers), 1H), 7.10-7.15 (m, 1H), |
| | | 7.19-7.21 (m, 1H), 7.49-7.53 (m, |
| | | 1H), 7.95-7.97 (m, 2H), |
| | | 8.14-8.18 (m, 2H), 9.15 (s, 1H), 9.54 (2m, |
| | | (rotational isomers), 1H, D₂O |
| | | exchangeable). Mass (m/z): |
| | | 525.2 (M⁺ + 1). |
| 22 | (S)—O-Methyl(3-(3-fluoro-4-(4-(6- | ¹H-NMR (DMSO-d₆): δ |
| | (trifluoromethyl)nicotinoyl) piperazin- | 2.98-3.08 (m, 4H), 3.48 (m, 2H), |
| | 1-yl)phenyl)-2-oxooxazolidin-5-yl)methylcarbamothioate | 3.76-3.82 (m, 5H), 3.88-3.93 ((2s |
| | | (rotational isomers), 3H), |
| | | 4.10-4.14 (m, 1H), 4.86-4.88 (2m, |
| | | (rotational isomers), 1H), |
| | | 7.07-7.12 (m, 1H), 7.19-7.21 (m, 1H), |
| | | 7.48-7.52 (m, 1H), 8.01-8.03 (m, |
| | | 1H); 8.18-8.20 (m, 1H), 8.86 (s, |
| | | 1H), 9.46-9.54 (2m, (rotational |
| | | isomers), 1H, D₂O exchangeable). |
| | | Mass (m/z): 542.2 (M⁺ + 1). |
| 23 | (S)—O-Methyl(3-(3-fluoro-4-(4-(1-p- | ¹H-NMR (DMSO-d₆): δ |
| | tolylcyclopropanecarbonyl) piperazin- | 1.12-1.31 (m, 4H), 2.26 (s, 3H), |
| | 1-yl)phenyl)-2-oxooxazolidin-5-yl)methylcarbamothioate | 3.02-3.13 (m, 4H), 3.52 (m, 2H), |
| | | 3.73-3.77 (m, 5H), 3.87-3.92 ((2s |
| | | (rotational isomers), 3H), |
| | | 4.08-4.10 (m, 1H), 4.88 (2m, (rotational |
| | | isomers), 1H), 6.98-7.07 (m, 3H), |
| | | 7.12-7.16 (m, 3H), 7.45-7.48 (m, |
| | | 1H), 9.44-9.55 (2m, (rotational |
| | | isomers), 1H, D₂O exchangeable). |
| | | Mass (m/z): 527.2 (M⁺ + 1). |
| 24 | (S)—O-Methyl(3-(4-(4-(2,6-dichloro-5- | ¹H-NMR (DMSO-d₆): δ |
| | fluoronicotinoyl)piperazin-1-yl)-3- | 2.93-3.07 (m, 4H), 3.49 (m, 2H), |
| | fluorophenyl)-2-oxooxazolidin-5-yl)methylcarbamothioate | 3.74-3.93 (m, 8H), 4.10-4.14 (m, 1H), |
| | | 4.86-4.90 (2m (rotational isomers), |
| | | 1H), 7.08-7.20 (m, 2H), |
| | | 7.48-7.52 (m, 1H), 8.29-8.31 (m, 1H), |
| | | 9.54 (2m (rotational isomers), 1H, |
| | | D₂O exchangeable). Mass (m/z): |
| | | 559 (M⁺ − 1). |
| 25 | (S)—O-Methyl(3-(4-(4-(1H-indazole-3- | ¹H-NMR (DMSO-d₆): δ |
| | carbonyl)piperazin-1-yl)-3-fluorophenyl)- | 3.05-3.08 (m, 4H), 3.41 (m, 2H), |
| | 2-oxooxazolidin-5-yl)methyl | 3.74-3.77 (m, 5H), 3.88-3.93 ((2s (rotational |
| | carbamothioate | isomers), 3H), 4.10-4.19 (m, 1H), |
| | | 4.88 (2m (rotational isomers), 1H), |
| | | 7.12-7.25 (m, 3H), 7.43-7.63 (m, |
| | | 3H), 7.99-8.01 (m, 1H), |
| | | 9.46-9.56 (2m (rotational isomers), 1H, D₂O |
| | | exchangeable), 13.56 (s, 1H, D₂O |
| | | exchangeable). Mass (m/z): |
| | | 512.9 (M⁺). |
| 26 | (S)—O-Methyl(3-(3-fluoro-4-(4-(6- | ¹H-NMR (DMSO-d₆): δ 2.99 (m, |
| | hydroxynicotinoyl)piperazin-1-yl)phenyl)- | 4H), 3.66 (m, 4H), 3.74-3.82 (m, |
| | 2-oxooxazolidin-5-yl) methyl | 3H), 3.88-3.93 ((2s (rotational |
| | carbamothioate | isomers), 3H), 4.10-4.14 (m, 1H), |
| | | 4.88 (2m (rotational isomers), 1H), |
| | | 6.34-6.37 (d, 1H), 7.06-7.10 (m, |
| | | 1H), 7.18-7.20 (m, 1H), |
| | | 7.48-7.54 (m, 2H), 7.61-7.62 (m, 1H), |
| | | 9.52-9.53 (2m (rotational isomers), 1H, |
| | | D₂O exchangeable), 12.01 (s, 1H, |
| | | D₂O exchangeable). Mass (m/z): |
| | | 489.9 (M⁺). |
| 27 | (S)—O-Methyl(3-(4-(4-(benzo [d]thiazole- | ¹H-NMR (DMSO-d₆): δ 3.03 (m, |
| | 6-carbonyl) piperazin-1-yl)-3- | 4H); 3.49-3.55 (m, 2H), |
| | fluorophenyl)-2-oxo oxazolidin-5- | 3.74-3.82 (m, 5H), 3.88-3.93 ((2s |
| | yl)methylcarbamothioate | (rotational isomers), 3H), |
| | | 4.10-4.12 (m, 1H), 4.88 (2m (rotational |
| | | isomers), 1H), 7.11-7.21 (m, 2H), |
| | | 7.48-7.62 (m, 2H), 8.15-8.17 (m, |
| | | 1H), 8.32 (s, 1H), 9.52 (s, 1H), |
| | | 9.54-9.55 (2m (rotational isomers), |
| | | 1H, D₂O exchangeable). Mass |
| | | (m/z): 529.8 (M⁺). |
| 28 | (S)—O-Methyl(3-(3-fluoro-4-(4- | ¹H-NMR (DMSO-d₆): δ 2.65 (m, |
| | (thiazolidine-2-carbonyl)piperazin-1- | 1H), 2.95-2.99 (m, 4H), |
| | yl)phenyl)-2-oxooxazolidin-5-yl)methylcarbamothioate | 3.11-3.13 (m, 3H), 3.65-3.93 (m, 11H), |
| | | 4.35 (m, 1H), 4.88-4.90 (2m |
| | | (rotational isomers), 1H), 5.18 (d, |
| | | 1H, D₂O exchangeable), |
| | | 7.06-7.11 (m, 1H), 7.18-7.20 (m, 1H), |
| | | 7.48-7.52 (m, 1H), 9.46-9.54 (2m |
| | | (rotational isomers), 1H, D₂O |
| | | exchangeable). Mass (m/z): |
| | | 484.9 (M⁺ + 1). |
| 29 | (S)—O-Methyl(3-(4-(4-(1H-benzo[d]imidazole- | ¹H-NMR (DMSO-d₆): δ 3.01 (m, |
| | 5-carbonyl)piperazin-1-yl)- | 4H), 3.48-3.82 (m, 7H), |
| | 3-fluorophenyl)-2-oxooxazolidin-5-yl)methyl | 3.88-3.93 (2s (rotational isomers), 3H), |
| | carbamothioate | 4.10-4.14 (m, 1H), 4.88 (2m |
| | | (rotational isomers), 1H), |
| | | 7.09-7.29 (m, 3H), 7.48-7.66 (m, 3H), |
| | | 8.32 (s, 1H), 9.46-9.54 (2m |
| | | (rotational isomers), 1H, D₂O |
| | | exchangeable), 12.65-12.79 (s, 1H, |
| | | D₂O exchangeable). Mass (m/z): |
| | | 513.1 (M⁺ + 1). |
| 30 | (S)—O-Methyl (3-(4-(4-(5- | ¹H-NMR (DMSO-d₆): δ 3.06 (m, |
| | chlorobenzofuran-2-carbonyl)piperazin- | 4H), 3.50 (m, 2H), 3.75-3.82 (m, |
| | 1-yl)-3-fluorophenyl)-2- | 5H), 3.88-3.93 ((2s (rotational |
| | oxooxazolidin-5-yl)methyl | isomers), 3H), 4.11-4.15 (m, 1H), |
| | carbamothioate | 4.89-4.90 (2m (rotational isomers), |
| | | 1H), 7.08-7.21 (m, 2H), |
| | | 7.43-7.54 (m, 3H), 7.73-7.84 (m, 2H), |
| | | 9.47-9.55 (2m (rotational isomers), |
| | | 1H, D₂O exchangeable). Mass |
| | | (m/z): 547.0 (M⁺). |
| 31 | (S)—O-Methyl(3-(3-fluoro-4-(4-((R)- | ¹H-NMR (DMSO-d₆): δ 2.65 (m, |
| | thiazolidine-4-carbonyl) piperazin-1- | 1H), 2.95-2.99 (m, 4H), |
| | yl)phenyl)-2-oxooxazolidin-5- | 3.11-3.16 (m, 3H), 3.65-4.12 (m, 12H), |
| | yl)methylcarbamothioate | 4.36 (m, 1H), 4.88-4.90 (2m (rotational |
| | | isomers), 1H), 7.07-7.11 (m, 1H), |
| | | 7.18-7.20 (m, 1H), 7.48-7.53 (m, |
| | | 1H), 9.45-9.54 (2m (rotational |
| | | isomers), 1H, D₂O exchangeable). |
| | | Mass (m/z): 484.1 (M⁺ + 1). |
| 32 | (S)—O-Methyl (3-(3-fluoro-4-(4-(2,2,2- | ¹H-NMR (DMSO-d₆): δ |
| | trifluoroacetyl)piperazin-1-yl)phenyl)- | 3.05-3.06 (m, 4H), 3.51 (m, 2H), |
| | 2-oxooxazolidin-5-yl)methyl | 3.74-3.82 (m, 5H), 3.88-3.93 (2s |
| | carbamothioate | (rotational isomers), 3H), |
| | | 4.07-4.15 (m, 1H), 4.87-4.90 (2m |
| | | (rotational isomers), 1H), |
| | | 7.09-7.13 (m, 1H), 7.18-7.21 (m, 1H), |
| | | 7.49-7.53 (m, 1H), 9.46-9.54 (2m |
| | | (rotational isomers), 1H, D₂O |
| | | exchangeable). Mass (m/z): |
| | | 464.9 (M⁺ + 1). |
| 33 | O-Methyl ((5S)-3-(4-(4-(2,2-difluoro | ¹H-NMR (DMSO-d₆): δ |
| | cyclopropanecarbonyl)piperazin-1-yl)- | 1.86-1.95 (m, 2H), 2.93-2.97 (m, 3H), |
| | 3-fluorophenyl)-2-oxooxazolidin-5- | 3.08 (m, 1H), 3.18-3.20 (m, 1H), |
| | yl)methyl carbamothioate | 3.62 (m, 4H), 3.75-3.80 (m, 3H), |
| | | 3.82-3.92 (2s (rotational isomers), |
| | | 3H), 4.10-4.15 (m, 1H), 4.89 (2m |
| | | (rotational isomers), 1H), |
| | | 7.07-7.12 (m, 1H), 7.18-7.20 (m, 1H), |
| | | 7.49-7.51 (m, 1H), 9.52-9.53 (2m |
| | | (rotational isomers), 1H, D₂O |
| | | exchangeable). Mass (m/z): |
| | | 472.8 (M⁺). |
| 34 | (S)—O-Methyl (3-(4-(4-(4-amino | ¹H-NMR (DMSO-d₆): δ-3.00 (bs, |
| | nicotinoyl)piperazin-1-yl)-3-fluorophenyl)- | 4H); 3.47-3.50 (m, 4H), 3.61 (m, |
| | 2-oxooxazolidin-5-yl)methylcarbamothioate | 2H), 3.88 (m, 1H), 3.93 (2s |
| | | (rotational isomers), 3H), |
| | | 4.10-4.14 (m, 1H), 4.88 (2m (rotational |
| | | isomers) 1H), 6.17 (bs, 2H, D₂O |
| | | exchangeable), 6.59-6.60 (m, 1H), |
| | | 7.07-7.11 (m, 1H), 7.18-7.20 (m, |
| | | 1H), 7.48-7.51 (m, 1H), 8.02 (s, |
| | | 2H), 9.54 (2m (rotational isomers), |
| | | 1H, D₂O exchangeable). Mass |
| | | (m/z): 488.8 (M⁺). |
| 35 | (S)—O-Methyl (3-(3-fluoro-4-(4-(6- | ¹H-NMR (DMSO-d₆): δ |
| | fluoronicotinoyl)piperazin-1-yl)phenyl)- | 3.11-3.17 (m, 4H), 3.46 (m, 3H), |
| | 2-oxooxazolidin-5-yl)methyl | 3.61-3.63 (m, 4H), 3.86-3.92 (2s |
| | carbamothioate | (rotational isomers), 3H), |
| | | 4.22-4.25 (m, 1H), 4.99-5.00 (2m |
| | | (rotational isomers), 1H), |
| | | 7.20-7.24 (m, 1H), 7.31-7.33 (m, 1H), |
| | | 7.41-7.42 (m, 1H), 7.61-7.64 (m, |
| | | 1H), 8.20-8.24 (m, 1H), 8.50 (s, |
| | | 1H), 9.65 (2m (rotational isomers), |
| | | 1H, D₂O exchangeable). Mass |
| | | (m/z): 491.8 (M⁺). |
| 36 | (S)—O-Methyl (3-(4-(4-(2-amino-4- | ¹H-NMR (DMSO-d₆): δ 2.96 (m, |
| | (trifluoromethyl)thiazole-5-carbonyl)piperazin- | 4H), 3.49-3.58 (m, 4H), |
| | 1-yl)-3-fluorophenyl)-2- | 3.75-3.82 (m, 3H), 3.93 (2s (rotational |
| | oxooxazolidin-5-yl)methyl | isomers), 3H), 4.10-4.14 (m, 1H), |
| | carbamothioate | 4.85-4.89 (2m (rotational isomers), |
| | | 1H), 7.07-7.10 (m, 1H), |
| | | 7.16-7.20 (m, 1H), 7.48-7.52 (m, 1H), |
| | | 7.73 (s, 2H, D₂O exchangeable), |
| | | 9.52-9.53 (2m (rotational isomers) |
| | | 1H, D₂O exchangeable. Mass |
| | | (m/z): 562.7 (M⁺). |
| 37 | (S)—O-Methyl (3-(4-(4-(6-amino | ¹H-NMR (DMSO-d₆): δ 2.99 (m, |
| | nicotinoyl)piperazin-1-yl)-3-fluorophenyl)- | 4H), 3.66 (m, 4H), 3.75-3.77 (m, |
| | 2-oxooxazolidin-5-yl)methyl | 3H), 3.88-3.93 (2s (rotational |
| | carbamothioate | isomers), 3H), 4.10-4.14 (m, 1H), |
| | | 4.85-4.90 (2m (rotational isomers), |
| | | 1H), 6.40 (s, 2H, D₂O |
| | | exchangeable), 6.43-6.44 (m, 1H) |
| | | 7.06-7.12 (m, 1H), 7.18-7.20 (m, |
| | | 1H), 7.47-7.52 (m, 2H), 8.05 (s, |
| | | 1H), 9.52-9.55 (2m (rotational |
| | | isomers), 1H, D₂O exchangeable). |
| | | Mass (m/z): 489.8 (M⁺ + 1). |
| 38 | (S)—O-Methyl (3-(3-fluoro-4-(4-(5- | ¹H-NMR (DMSO-d₆): δ |
| | fluoronicotinoyl)piperazin-1-yl)phenyl)- | 2.95-3.06 (m, 4H), 3.41 (m, 2H), |
| | 2-oxooxazolidin-5-yl)methyl | 3.76-3.82 (m, 5H), 3.88-3.93 (2s |
| | carbamothioate | (rotational isomers), 3H), |
| | | 4.10-4.12 (m, 1H), 4.88-4.92 (2m |
| | | (rotational isomers, 1H), |
| | | 7.10-7.13 (m, 1H), 7.19-7.21 (m, 1H), |
| | | 7.49-7.52 (m, 2H), 8.06-8.08 (m, |
| | | 1H), 8.35-8.36 (m, 1H), 9.53 (2m |
| | | (rotational isomers), 1H, D₂O |
| | | exchangeable). Mass (m/z): |
| | | 492.8 (M⁺ + 1). |
| 39 | (S)—O-Methyl (3-(3-fluoro-4-(4-(5- | ¹H-NMR (DMSO-d₆): δ |
| | methylisoxazole-3-carbonyl)piperazin- | 2.47-2.51 (m, 3H) 3.00-3.06 (m, 4H), |
| | 1-yl)phenyl)-2-oxooxazolidin-5-yl)methylcarbamothioate | 3.72-3.80 (m, 7H), 3.88-3.93 (2s |
| | | (rotational isomers), 3H), |
| | | 4.10-4.15 (m, 1H), 4.88-4.90 (2m |
| | | (rotational isomers), 1H), 6.49 (s, |
| | | 1H), 7.08-7.13 (m, 1H), |
| | | 7.18-7.20 (m, 1H), 7.48-7.53 (m, 1H) |
| | | 9.52-9.55 (2m (rotational isomers), |
| | | 1H, D₂O exchangeable. Mass |
| | | (m/z): 477.8 (M⁺). |

### Example 40

### Synthesis of (S)—O-methyl(3-(3-fluoro-4-(4-(3-(5-nitrofuran-2-yl)acryloyl)piperazin-1-yl)phenyl)-2-oxooxazolidin-5 yl)methylcarbamothioate

Pyridine (2 ml) and piperidine (0.5 ml) was added to a mixture of 5-nitrofurfural (1.0 g, 7.1 mmol) and malonic acid (1.5 g, 14.1 mmol) and the reaction mixture was refluxed for 1 hour, subsequently it was cooled to room temperature, poured into water and the pfl was adjusted to 2 using cone HCl. The precipitated solid was filtered and dried to obtain 3-(5-nitrofuran-2-yl)acrylic acid. The acid (0.29 g, 1.56 mmol) thus obtained was then used to couple with (S)—O-methyl(3-(3-fluoro-4-(piperazin-1-yl)phenyl)-2-oxooxazolidin-5-yl)methyl carbamothioate (0.51 g, 1.3 mmol), prepared according to the procedure described in the step 3 of example 1 in DMF (5 ml) in presence of 1-hydroxybenztriazole hydrate (HOBt) (0.18 g, 1.3 mmol), 1-ethyl-3-(3-dimethylaminopropyl) carbodiimide hydrochloride (EDC) (0.5 g, 2.6 mmol) and triethyl amine (0.5 ml, 3.9 mmol) to obtain the title compound (0.12 g, 17.2%). ¹H-NMR (DMSO-d₆): δ 3.01 (m, 4H), 3.75-3.82 (m, 7H), 3.88-3.93 (2 s (rotational isomers), 3H), 4.10-4.15 (m, 1H), 4.88-4.89 (2 m, (rotational isomers), 1H), 7.07-7.12 (m, 1H), 7.18-7.21 (m, 1H), 7.27-7.28 (m, 1H), 7.32-7.36 (m, 2H), 7.40-7.53 (m, 1H), 7.78-7.79 (m, 1H), 9.45-9.54 (2 m, (rotational isomers), 1H, D₂O exchangeable). Mass (m/z): 534.1 (M⁺+1).

### Example 41

### Synthesis of (S)—O-methyl (3-(4-(4-(2-aminoacetyl)piperazin-1-yl)-3-fluorophenyl)-2-oxooxazolidin-5-yl)methylcarbamothioate hydrochloride

(S)—O-Methyl(3-(3-fluoro-4-(piperazin-1-yl)phenyl)-2-oxooxazolidin-5-yl)methyl carbamothioate (0.63 g, 3.6 mmol) prepared according to the procedure described in the step 3 of example 1, and N-boc-glycine (037 g, 2.10 mmol) were dissolved in DMF (5 ml) along with 1-hydroxybenztriazole hydrate (HOBt) (0.22 g, 1.8 mmol), 1-ethyl-3-(3-dimethylaminopropyl) carbodiimide hydrochloride (EDC) (0.63 g, 3.6 mmol) and triethyl amine (0.7 ml, 5.4 mmol) as described in the procedure in example 1 to obtain (S)-t-butyl 2-(4-(2-fluoro-4-(5-((methoxycarbonothioylamino)methyl)-2-oxooxazolidin-3-yl)phenyl)piperazin-1-yl)-2-oxoethylcarbamate which on treatment with dioxane-HCl (10 ml) gave the title compound (0.25 g, 32.67%). ¹H-NMR (DMSO-d₆): δ 2.98-3.00 (m, 4H), 3.67-3.70 (s, 4H), 3.75-3.81 (m, 4H), 3.88 (s, 2H), 3.92-3.93 (2 s (rotational isomers), 3H), 4.10-4.15 (m, 1H), 4.88-4.89 (2 m (rotational isomers), 1H), 7.07-7.10 (m, 1H), 7.19-7.22 (m, 1H), 7.49-7.53 (m, 1H), 8.11 (s, 2H, D₂O exchangeable) 9.55-9.57 (2 m (rotational isomer), 1H, D₂O exchangeable). Mass (m/z): 425.8 ((M⁺—Cl).

The following compound was prepared according to the procedure given in Example 41.

| | | |
|---|---|---|
| 42 | (S)—O-Methyl (3-(4-(4-((S)-2-amino-3- | ¹H-NMR (DMSO-d₆): δ |
| | hydroxypropanoyl)piperazin-1-yl)-3- | 2.94-3.01 (m, 4H), 3.46-3.82 (m, 10H), |
| | fluorophenyl)-2-oxooxazolidin-5- | 3.88-3.93 (2s (rotational isomers), |
| | yl)methylcarbamothioate hydrochloride | 3H), 4.10-4.15 (m, 1H), |
| | | 4.42-4.43 (m, 1H), 4.87-4.91 (2m |
| | | (rotational isomers), 1H), |
| | | 7.08-7.12 (m, 1H), 7.19-7.22 (m, 1H), |
| | | 7.49-7.53 (m, 1H), 8.15-8.16 (bs, |
| | | 3H, D₂O exchangeable), |
| | | 9.55-9.57 (2m (rotational isomers, 1H, D₂O |
| | | exchangeable). Mass (m/z): |
| | | 456.9 ((M⁺ + 1) − Cl). |

### Example 43

### Synthesis of (S)—O-methyl (3-(3-fluoro-4-(4-(2-(methylsulfonamido)acetyl)piperazin-1-yl)phenyl)-2-oxooxazolidin-5-yl)methylcarbamothioate

Methane sulfonyl chloride (0.03 ml, 0.35 mmol) was added dropwise to the cold (0° C.) mixture of compound 41 (0.15 g, 0.35 mmol) and triethylamine (0.08 ml, 0.60 mmol) in dichloromethane (10 ml) under stirring. Upon completion of reaction the reaction mass was poured into water and then the product was extracted with EtOAc. The organic layer was separated, dried over anhydrous Na₂SO₄ and the solvent was removed under vacuum to afford the title compound (0.03 g, 17.2%). ¹H-NMR (DMSO-d₆): δ 2.95-2.99 (m, 7H), 3.49-3.62 (m, 4H), 3.75-3.82 (m, 3H), 3.88 (2 s (rotational isomers), 3H), 3.96-3.97 (m, 2H), 4.10-4.14 (m, 1H), 4.87-4.88 (2 m (rotational isomers), 1H), 7.06-7.13 (m, 2H, 1H is D₂O exchangeable), 7.18-7.20 (m, 1H), 7.48-7.53 (m, 1H), 9.52-9.55 (2 m (rotational isomers), 1H, D₂O exchangeable).

Mass (m/z): 503.8 (M⁺).

### Example 44

### Synthesis of (S)—O-methyl (3-(3-fluoro-4-(4-(isoxazole-5-carbonyl)piperazin-1-yl)phenyl)-2-oxooxazolidin-5-yl)methylcarbamothioate

To a cold (0° C.) solution of O-methyl (S)—N-[3-[3-fluoro-4-[piperazin-1-yl]phenyl]-2-oxo-oxazolidin-5-ylmethyl]thiocarbamate (0.67 g, 1.8 mmol), prepared according to the procedure described in the step 3 of example 1 in dry DMF (5 ml), acid chloride (0.18 ml, 1.8 mmol) and triethylamine (0.8 ml, 5.4 mmol) was added and the stirring was continued at room temperature for 2 hours. The reaction mixture was diluted with ethyl acetate and washed with sodium bicarbonate solution, water and brine successively. The ethyl acetate layer was dried over anhydrous Na₂SO₄, concentrated to dryness and the crude product was purified by flash chromatography on silica gel using a Biotage SP-1 system (12+M) cartridge using ethyl acetate and hexane mixture as an eluent to afford the title compound (0.15 g, 20.4%). ¹H-NMR (DMSO-d₆): δ 3.04(m, 4H), 3.69 (m, 2H), 3.74-3.79 (m, 5H); 3.88-3.92 (2 s (rotational isomers), 3H), 4.10-4.14 (m, 1H), 4.86-4.90 (2 m, (rotational isomers), 1H), 6.981-6.983 (m, 1H), 7.08-7.20 (m, 2H), 7.49-7.52 (m, 1H); 8.77 (m, 1H); 9.44-9.54 (2 m, (rotational isomers), 1H, D₂O exchangeable). Mass (m/z): 464.1 (M⁺+1).

The following compounds were prepared according to the procedure given in Example 44.

| | | |
|---|---|---|
| 45 | (S)—O-Methyl (3-(4-(4-(2-(benzyloxy)acetyl)piperazin- | ¹H-NMR (DMSO-d₆): δ |
| | 1-yl)-3-fluorophenyl)-2- | 2.95 (brs, 4H), 3.56-3.93 (m, 11H), |
| | oxooxazolidin-5-yl)methylcarbamothioate | 4.24 (s, 2H), 4.53 (s, 2H), |
| | | 4.87-4.88 (2m, (rotational isomers), |
| | | 1H), 7.04-7.52 (m, 8H), |
| | | 9.52-9.55 (2m, (rotational isomers), |
| | | 1H, D₂O exchangeable). Mass |
| | | (m/z): 517.2 (M⁺ + 1). |
| 46 | (S)—O-Methyl(3-(4-(4-(cyclohexane | ¹H-NMR (DMSO-d₆): δ |
| | carbonyl)piperazin-1-yl)-3-fluorophenyl)- | 1.3-1.7 (m, 6H), 2.89-2.96 (m, 6H), |
| | 2-oxooxazolidin-5-yl)methyl | 3.59-3.92 (m, 13H), |
| | carbamothioate | 4.11-4.13 (m, 1H), 4.88-4.9 (2m, |
| | | (rotational isomers), 1H), |
| | | 7.06-7.52 (m, 3H), 9.51-9.54 (2m, |
| | | (rotational isomers), 1H, D₂O |
| | | exchangeable). Mass (m/z): |
| | | 479.2 (M⁺ + 1). |
| 47 | (S)—O-Methyl (3-(4-(4-(cyclopropane | ¹H-NMR (DMSO-d₆): δ |
| | carbonyl)piperazin-1-yl)-3-fluorophenyl)- | 0.74-0.76 (m, 4H), 2.01-2.02 (m, |
| | 2-oxooxazolidin-5-yl)methyl | 1H), 2.92 (br, 2H), 3.0 (brs, |
| | carbamothioate | 2H), 3.62 (brs, 2H), |
| | | 3.75-3.93 (m, 8H), 4.11-4.12 (m, 1H), |
| | | 4.88-4.89 (2m, (rotational |
| | | isomers), 1H), 7.07-7.52 (m, |
| | | 3H), 9.51-9.54 (2m, (rotational |
| | | isomers), 1H, D₂O |
| | | exchangeable). Mass: |
| | | 437.1 (M⁺ + 1). |
| 48 | (S)—O-Methyl(3-(4-(4-(cyclopentane | ¹H-NMR (DMSO-d₆): δ |
| | carbonyl)piperazin-1-yl)-3-fluorophenyl)- | 1.54-1.76 (m, 8H), 2.91-2.92 (m, 4H), |
| | 2-oxooxazolidin-5-yl)methyl | 2.96-2.97 (m, 1H), 3.40 (m, 2H), |
| | carbamothioate | 3.61-3.77 (m, 5H), 3.88-3.93 ((2s |
| | | (rotational isomers), 3H), |
| | | 4.12 (m, 1H), 4.86 (2m, |
| | | (rotational isomers), 1H), |
| | | 7.08-7.10 (m, 1H), 7.16-7.17 (m, 1H), |
| | | 7.47-7.52 (m, 1H), 9.53 (2m, |
| | | (rotational isomers), 1H, D₂O |
| | | exchangeable). Mass (m/z): |
| | | 465.2 (M⁺+ 1). |
| 49 | (S)—O-Methyl(3-(4-(4-(2-chloroacetyl)piperazin- | ¹H-NMR (DMSO-d₆): δ |
| | 1-yl)-3-fluorophenyl)-2-oxo | 2.95-3.00 (m, 4H), 3.61 (m, 4H), |
| | oxazolidin-5-yl)methylcarbamothioate | 3.75-3.80 (m, 3H), 3.88-3.93 (2s |
| | | (rotational isomers), 3H), |
| | | 4.10-4.12 (m, 1H), 4.43 (s, 2H), |
| | | 4.87-4.88 (2m, (rotational isomers), |
| | | 1H), 7.07-7.08 (m, 1H), |
| | | 7.17-7.20 (m, 1H), 7.48-7.52 (m, 1H), |
| | | 9.52-9.54 (2m, (rotational |
| | | isomers), 1H, D₂Oexchangeable) |
| | | Mass: 444.8 (M⁺). |

### Example 50

### Synthesis of (S)—S-methyl (3-(4-(4-(4-chlorobenzoyl)piperazin-1-yl)-3-fluorophenyl)-2-oxooxazolidin-5-yl)methylcarbamothioate

To a solution of (S)-t-butyl 4-(2-fluoro-4-(5-((methoxycarbonothioylamino) methyl)-2-oxooxazolidin-3-yl)phenyl)piperazine-1-carboxylate (0.5 g, 1.07 mmol), prepared according to the procedure described in the step 2 of example 1, in DCM (2 ml), trifluoroacetic acid (0.44 ml, 5.35 mmol) was added dropwise and the reaction mixture was stirred well for 12 hours. DCM was evaporated under reduced pressure, the residue was diluted with diethyl ether (15 ml), while a solid precipitated out, which was dried, filtered and dried under high vacuum. This precipitate (0.52 g, 1.07 mmol) was then suspended in dry DCM (5 ml) followed by the addition of triethylamine (0.5 ml, 3.21 mmol) and chlorobenzoyl chloride (0.2 ml, 1.6 mmol) under stirring at 0° C.

The reaction mixture was allowed to attain ambient temperature and the stirring was continued for 2 hours. The resulting reaction mixture was diluted with DCM (25 ml) and washed with sodium bicarbonate solution, water and brine. The organic layer obtained, was dried over anhydrous Na₂SO₄, concentrated to dryness and the residue was subjected to silica gel flash chromatography using a Biotage SP-1 system (12+M) cartridge using ethyl acetate and hexane as eluent to afford the title compound (0.2 g, 37.04%). ¹H-NMR (DMSO-d₆): δ 2.21(s, 3H), 3.03 (bs, 5H), 3.45-3.57(m, 3H), 3.70-3.77 (m, 3H), 4.09-4.11 (m, 1H), 4.73-4.74 (m, 1H), 7.07-7.12 (m, 1H), 7.17-7.19 (m, 1H), 7.46-7.54 (m, 5H), 8.53 (m, 1H, D₂O exchangeable). Mass (m/z): 507.1 (M⁺).

The following compounds were prepared according to the procedure given in Example 50.

| | | |
|---|---|---|
| 51 | (S)—S-Methyl (3-(3-fluoro-4-(4- | ¹H-NMR (DMSO-d₆): δ 2.21 (s, |
| | (morpholine-4-carbonyl) piperazin-1-yl)phenyl)- | 3H), 2.94-2.95 (bs, 5H), |
| | 2-oxooxazolidin-5-yl)methyl | 3.15-3.17 (m, 5H), 145-3.48 (m, 2H), |
| | carbamothioate | 3.56-3.58 (m, 5H), 3.71-3.73 (m, |
| | | 2H), 4.07-4.11 (m, 1H), 4.72 (m, |
| | | 1H), 7.05-7.10 (m, 1H), |
| | | 7.16-7.19 (m, 1H), 7.46-7.51 (m, 1H), |
| | | 8.53-8.60 (m, 1H, D₂O |
| | | exchangeable). Mass (m/z): |
| | | 482.1 (M⁺ + 1). |
| 52 | (S)—S-Methyl (3-(3-fluoro-4-(4-(4- | ¹H-NMR (DMSO-d₆): δ 2.21 (s, |
| | fluorobenzoyl)piperazin-1-yl)phenyl)-2- | 3H), 3.00 (bs, 5H), 3.43-3.50 (m, |
| | oxooxazolidin-5-yl)methylcarbamothioate | 3H), 3.70-3.73 (m, 3H), |
| | | 4.07-4.11 (m, 1H), 4.71-4.74 (m, 1H), |
| | | 7.07-7.09 (m, 1H), 7.12-7.17 (m, |
| | | 1H), 7.19-7.27 (m, 2H), |
| | | 7.31-7.53 (m, 3H), 8.54 (m, 1H, D₂O |
| | | exchangeable). Mass (m/z): |
| | | 491.1 (M⁺ + 1). |
| 53 | (S)—S-Methyl (3-(4-(4-(2-chloro | ¹H-NMR (DMSO-d₆): δ 2.21 (s, |
| | acetyl)piperazin-1-yl)-3-fluorophenyl)-2- | 3H), 2.95-3.00 (m, 2H), |
| | oxooxazolidin-5-yl)methylcarbamothioate | 3.31-3.33 (m, 2H), 3.45-3.47 (m, 2H), |
| | | 3.71-3.74 (m, 3H), 4.07-4.12 (m, |
| | | 3H), 4.43 (s, 2H), 4.72-4.73 (m, |
| | | 1H), 7.06-7.10 (m, 1H), |
| | | 7.17-7.20 (m, 1H), 7.48-7.52 (m, 1H), |
| | | 8.53-8.55 (m, 1H, D₂Oexchangeable); |
| | | Mass (m/z): 445 (M⁺) |
| 54 | (S)—S-Methyl (3-(4-(4-(3-chloro benzoyl)piperazin- | ¹H-NMR (DMSO-d₆): δ 2.21 (s, |
| | 1-yl)-3-fluorophenyl)-2- | 3H), 2.96-3.04 (m, 4H), |
| | oxooxazolidin-5-yl)methyl | 3.39-3.46 (m, 4H), 3.71-3.77 (m, 3H), |
| | carbamothioate | 4.07-4.11 (m, 1H), 4.72 (bs, 1H), |
| | | 7.07-7.12 (m, 1H), 7.17-7.19 (m, |
| | | 1H), 7.39-7.41 (m, 1H), |
| | | 7.47-7.55 (m, 4H), 8.55 (m, 1H, D₂O |
| | | exchangeable). Mass (m/z): |
| | | 507.1 (M⁺) |
| 55 | (S)—S-Methyl (3-(3-fluoro-4-(4-pivaloyl | ¹H-NMR (DMSO-d₆): δ 1.22 (s, |
| | piperazin-1-yl)phenyl)-2-oxo oxazolidin- | 9H), 2.21 (s, 3H), 2.94 (bs, 4H), |
| | 5-yl)methylcarbamothioate | 3.70-3.74 (m, 7H), 4.07-4.12 (m, |
| | | 1H), 4.71-4.74 (m, 1H), |
| | | 7.06-7.10 (m, 1H), 7.17-7.19 (m, 1H), |
| | | 7.47-7.51 (d, 1H), 8.55 (m, 1H, D₂O |
| | | exchangeable). Mass (m/z): |
| | | 453.1 (M⁺ + 1) |

### Example 56

### (S)—S-Methyl(3-(3-fluoro-4-(4-(morpholine-4-carbonothioyl)piperazin-1-yl)phenyl)-2-oxooxazolidin-5-yl)methylcarbamothioate

To a solution of (S)-t-butyl-4-(2-fluoro-4-(5-((methoxycarbonothioylamino) methyl)-2-oxooxazolidin-3-yl)phenyl)piperazine-1-carboxylate (0.48 g, 1.03 mmol), prepared according to the procedure described in the step 2 of example 1, in DCM (5 ml), trifluoroacetic acid (0.5 ml, 6.0 mmol) was added drop wise and stirred well for 12 hours. DCM was evaporated under reduced pressure and the residue was triturated with diethyl ether (15 ml) and the precipitate obtained was filtered and dried under high vacuum. The precipitate (0.49 g, 1.03 mmol) was then suspended in DCM (5 ml) and the reaction mixture and it was cooled to 10° C., into which triethyl amine (0.6 ml, 4.28 mmol) and thiophosgene (0.08 ml, 1.03 mmol) were added under stirring over 30 minutes. The reaction mass was allowed to attain ambient temperature and was further stirred for another 90 minutes. Morpholine (0.3 ml, 3.21 mmol) was added into the reaction mixture and was allowed to stir for another 15 hours. On completion of the reaction, the resulting mixture was diluted with DCM (30 ml) and washed with water wash thrice. The organic layer was separated, dried over anhydrous Na₂SO₄ and solvent was removed under vacuum to yield a yellow residue. The crude product obtained was purified by flash chromatography on silica gel using a Biotage SP-1 system (12+M) cartridge using ethyl acetate and hexane as eluent to afford the title compound (0.04 g, 8.0%). ¹H-NMR (DMSO-d₆): δ 2.21 (s, 3H), 3.03 (m, 4H), 3.16-3.17 (m, 2H), 3.53-3.74 (m, 12H), 4.04-4.11(m, 2H), 4.73 (m, 1H), 7.07-7.11 (m, 1H), 7.17-7.19 (m, 1H), 7.48-7.52 (d, 1H), 8.57 (m, 1H, D₂O exchangeable). Mass (m/z): 498.1 (M⁺+1).

### Example 57

### (S)—S-Methyl (3-(4-(4-(2-(2,5-dichlorophenylthio)acetyl)piperazin-1-yl)-3-fluorophenyl)-2-oxooxazolidin-5-yl)methylcarbamothioate

Compound 53 (0.25 g, 0.56 mmol) was dissolved in DMF (2 ml) and the reaction mixture was cooled to −20° C. into which 2,6-lutidine (0.28 ml, 0.67 mmol) was added dropwise and stirred for 10 minutes. 2,5-dichlorothiophenol (0.12 g, 0.67 mmol) was added into the chilled mixture and it was stirred for another 30 minutes, subsequently the temperature of the reaction mixture was raised to 0° C. and stirred for another 3 hours. After completion of reaction, the mixture was poured into 100 ml of ice-cold water and stirred for 10 minutes. The white precipitate thus obtained was filtered, dried and purified by flash chromatography on silica gel using a Biotage SP-1 system (12+M) cartridge using ethyl acetate and hexane as eluent to afford the title compound (0.042 g, 13%). ¹H-NMR (DMSO-d₆): δ 2.21(s, 3H), 2.95 (m, 2H), 3.04 (m, 2H), 339(m, 2H), 3.47 (m, 2H), 3.64 (m, 2H), 330-3.74 (m, 3H), 4.08-4.12 (m, 1H), 4.26 (s, 2H), 4.71-4.75 (m, 1H), 7.06-7.26 (m, 3H), 7.47-7.56 (m, 3H), 8.55 (m, 1H, D₂O exchangeable). Mass (m/z): 588 (M⁺+1)

### Example 58

### (S)—S-Methyl (3-(4-(4-(imidazole-1-carbonyl)piperazin-1-yl)-3-fluoro phenyl)-2-oxooxazolidin-5-yl)methylcarbamothioate

To a solution of (S)-t-butyl-4-(2-fluoro-4-(5-((methoxycarbonothioylamino) methyl)-2-oxooxazolidin-3-yl)phenyl)piperazine-1-carboxylate (0.7 g, 1.49 mmol), prepared according to the procedure described in the step 2 of example 1) in DCM (5 ml), trifluoroacetic acid (0.50 ml, 6 mmol) was added drop wise and stirred well for 12 hours. DCM was evaporated under reduced pressure and the residue was diluted with diethyl ether (15 ml) and the precipitate obtained was dried under high vacuum. The above-mentioned precipitate was then suspended in DCM (5 ml) and cooled to 0° C., into which triethyl amine (0.65 ml, 4.5 mmol) and 1,1′-carbonyl diimidazole (0.32 g, 1.95 mmol) were added under stirring. The stirring was continued for 4 hours at ambient temperature. Once TLC confirmed the completion of the reaction, the resulting mixture was diluted with DCM (50 ml) and was given water wash thrice. The organic layer was separated, dried over Na₂SO₄ and solvent was removed under vacuum to yield a pale yellow residue. The crude product was purified by flash chromatography on silica gel using a Biotage SP-1 system (12+M) cartridge using ethyl acetate and hexane as eluent to afford the title compound (0.08 g, 11.6%). ¹H-NMR (DMSO-d₆): δ 2.21(s, 3H), 3.06-3.08 (m, 4H), 3.45-3.48 (m, 2H), 3.65-3.67 (m, 4H), 3.70-3.74 (m, 1H), 4.08-4.12 (m, 1H), 4.72-4.74 (m, 1H), 7.05-7.21(m, 3H), 7.48-7.53 (m, 2H), 8.07 (s, 1H), 8.54 (m, 1H, D₂O exchangeable). Mass (m/z): 463.1 (M⁺+1).

### Example 59

### (S)—S-methyl (3-(3-fluoro-4-(4-(1-methyl-indazole-3-carbonyl)piperazin-1-yl)phenyl)-2-oxooxazolidin-5-yl)methylcarbamothioate

To a solution of (S)-t-butyl-4-(2-fluoro-4-(5-((methoxycarbonothioylamino) methyl)-2-oxooxazolidin-3-yl)phenyl)piperazine-1-carboxylate (0.5 g, 1.07 mmol) (prepared according to the procedure described in the step 2 of example 1) in DCM (5 ml), trifluoroacetic acid (0.4 ml, 4.28 mmol) was added drop wise and stirred well for 12 hours. DCM was evaporated under reduced pressure and the residue was washed with diethyl ether (15 ml) and the precipitate obtained was dried under high vacuum. This precipitate (0.52 g, 1.07 mmol) was then dissolved in dry THF (5 ml) into which N-methyl indazole-3-carboxylic acid (0.19 g, 1.07 mmol), 1-hydroxybenztriazole hydrate (HOBO (0.03 g, 0.22 mmol), benzotriazole-1-yl-oxy-tris-(dimethylamino)-phosphoniumhexafluoro phosphate (BOP) (0.57 g, 1.28 mmol) and Diisopropylethylamine (DIPEA) (0.9 ml, 5.22 mmol) were added under stirring. The resulted reaction mass was stirred until completion of the reaction which was confirmed by TLC after 15 hours. The reaction mass was then poured into crushed ice. Semisolid residue obtained after decanting the aqueous supernatent liquid was then dissolved in DCM followed by brine wash. The organic layer was separated, dried over Na₂SO₄ and the solvent was removed under vacuum. The crude reaction material was purified by flash chromatography on silica gel using a Biotage SP-1 system (12+M) cartridge using ethyl acetate and hexane as eluent to afford the title compound (0.03 g, 6.1%). ¹H-NMR (DMSO-d₆): δ 2.1 (s, 3H), 3.06 (bs, 4H), 3.46-3.47 (m, 2H), 3.88 (m, 3H), 4.10-4.19 (m, 6H), 4.73 (m, 1H), 7.10-7.19 (m, 2H), 7.26 (m, 1H), 7.46-7.53 (m, 2H), 7.72-7.75 (m, 1H), 7.98-8.00 (m, 1H), 8.55 (m, 1H, D₂O exchangeable). Mass (m/z): 527.1 (M⁺+1).

The following compounds were prepared according to the procedure given in Example 59.

| | | |
|---|---|---|
| 60 | (S)—S-Methyl (3-(4-(4-(2-cyanoacetyl)piperazin- | ¹H-NMR (DMSO-d₆): δ 2.2 (s, |
| | 1-yl)-3-fluorophenyl)-2-oxo | 3H), 2.94-3.00 (m, 4H), |
| | oxazolidin-5-yl)methylcarbamothioate | 3.40-3.46 (m, 4H), 3.49-3.50 (d, 2H), |
| | | 3.60-3.61 (m, 1H), 3.88-4.11 (m, |
| | | 3H), 4.70-4.74 (s, 1H), |
| | | 7.05-7.09 (m, 1H), 7.17-7.20 (m, 1H), |
| | | 7.48-7.52 (m, 1H), 8.55 (m, 1H, |
| | | D₂O exchangeable). Mass (m/z): |
| | | 436.1 (M⁺ + 1). |
| 61 | (S)—S-Methyl(3-(4-(4-(5-chlorothiophene- | ¹H-NMR (DMSO-d₆): δ 2.1 (s, |
| | 2-carbonyl)piperazin-1-yl)-3-fluorophenyl)- | 3H), 3.03 (bs, 4H), 3.47 (m, 2H), |
| | 2-oxooxazolidin-5-yl)methyl | 3.62-3.88 (m, 5H), 4.08-4.12 (m, |
| | carbamothioate | 1H), 4.73-4.74 (m, 1H), |
| | | 7.07-7.12 (m, 1H), 7.18-7.19 (m, 2H), |
| | | 7.37-7.38 (d, 1H), |
| | | 7.49-7.53 (m, 1H), 8.55 (m, 1H, D₂O |
| | | exchangeable). Mass (m/z): |
| | | 513 (M⁺). |
| 62 | (S)—S-Methyl (3-(4-(4-(2-amino | ¹H-NMR (DMSO-d₆): δ 3.00 (bs, |
| | nicotinoyl) piperazin-1-yl)-3-fluorophenyl)- | 4H), 3.39-3.60 (m, 5H), |
| | 2-oxooxazolidin-5-yl)methyl | 3.74-3.77 (m, 3H), 3.88-3.93 (m, 3H), |
| | carbamothioate; | 4.12 (m, 1H), 6.00 (s, 2H, D₂O |
| | | exchangeable), 6.58-6.61 (m, |
| | | 1H), 7.08-7.10 (m, 1H), 7.17 (m, |
| | | 1H), 7.40-7.41 (m, 1H), |
| | | 7.47-7.50 (m, 1H), 8.00 (m, 1H), |
| | | 9.53 (m, 1H, D₂O exchangeable). |
| | | Mass (m/z): 489.1 (M⁺ + 1). |
| 63 | (S)—S-Methyl (3-(3-fluoro-4-(4-(6-fluoro- | ¹H-NMR (DMSO-d₆): δ 2.21 (s, |
| | 2-naphthoyl)piperazin-1-yl)phenyl)-2-oxo | 3H), 3.04 (bs, 4H), 3.41-3.47 (m, |
| | oxazolidin-5-yl)methylcarbamothioate | 2H), 3.56 (m, 2H), 3.70-3.74 (m, |
| | | 2H), 3.82 (bs, 2H), 4.07-4.12 (m, |
| | | 1H), 4.72 (m, 1H), 7.09-7.20 (m, |
| | | 2H), 7.49-7.51 (m, 1H), 7.54 (d, |
| | | 1H), 7.59-7.62 (d, 1H), |
| | | 7.78-7.80 (m, 1H), 7.99-8.01 (m, 1H, |
| | | 8.09-8.14 (m, 1H), 8.55 (m, 1H, |
| | | D₂O exchangeable). Mass (m/z): |
| | | 541.1 (M⁺ + 1). |
| 64 | (S)—S-Methyl (3-(4-(4-(2,2-difluoro | ¹H-NMR (DMSO-d₆): δ |
| | cyclopropanecarbonyl)piperazin-1-yl-)-3- | 1.87-1.95 (m, 2H), 2.22 (s, 3H), |
| | fluorophenyl)-2-oxooxazolidin-5-yl)methylcarbamothioate | 2.91-2.98 (m, 3H), 3.10-3.14 (m, 2H), |
| | | 3.49 (m, 2H), 3.65-3.78 (m, 5H), |
| | | 4.07-4.12 (m, 1H), 4.74 (bs, 1H), |
| | | 7.09-7.19 (m, 2H), 7.47-7.50 (m, |
| | | 1H), 8.54 (m, 1H, D₂O |
| | | exchangeable). Mass (m/z): |
| | | 473.1 (M⁺ + 1). |
| 65 | (S)—S-Methyl(3-(3-fluoro-4-(4-(6-fluoro | ¹H-NMR (DMSO-d₆): δ 2.21 (s, |
| | picolinoyl)piperazin-1-yl)phenyl)-2-oxo | 3H), 3.05 (bs, 2H), 3.27 (bs, 2H), |
| | oxazolidin-5-yl)methyl carbamothioate | 3.42-3.47 (m, 3H), |
| | | 3.54-3.80 (m, 4H), 4.09-4.11 (m, 1H), |
| | | 4.73 (m, 1H), 7.08-7.12 (m, 1H), |
| | | 7.17-7.19 (m, 1H), 7.31-7.33 (d, |
| | | 1H), 7.48-7.51 (d, 1H), |
| | | 7.57-7.59 (m, 1H), 8.12-8.16 (m, 1H), |
| | | 8.55 (m, 1H, D₂O exchangeable). |
| | | Mass (m/z): 492.1 (M⁺ + 1). |
| 66 | (S)—S-Methyl (3-(3-fluoro-4-(4-(5-methyl | ¹H-NMR (DMSO-d₆): δ 2.20 (s, |
| | isoxazole-3-carbonyl)piperazin-1-yl)phenyl)- | 3H), 2.50 (s, 3H), 2.98 (bs, 2H), |
| | 2-oxooxazolidin-5-yl)methyl | 3.04 (bs, 2H), 3.41-3.46 (m, 2H), |
| | carbamothioate | 3.72 (m, 3H), 3.80 (m, 2H), |
| | | 4.07-4.12 (m, 1H), 4.72 (m, 1H), |
| | | 6.49 (s, 1H), 7.08-7.12 (m, 1H), |
| | | 7.17-7.19 (m, 1H), 7.48-7.52 (m, |
| | | 1H), 8.55 (m, 1H, D₂O |
| | | exchangeable). Mass (m/z): |
| | | 478.1 (M⁺ + 1). |
| 67 | (S)—S-Methyl(3-(4-(4-(4-aminonicotinoyl)piperazin- | ¹H-NMR (DMSO-d₆): δ 2.21 (s, |
| | 1-yl-3-fluoropheny1)-2-oxo | 3H), 2.90-3.01 (bs, 4H), |
| | oxazolidin-5-yl)methyl carbamothioate | 3.42-3.73 (m, 7H), 4.03-4.11 (m, 1 H), |
| | | 4.72 (m, 1 H), 6.23 (s, 2H, D₂0 |
| | | exchangeable), 6.60-6.62 (m, |
| | | 1H), 7.06-7.10 (m, 2H), |
| | | 7.47-7.51 (m, 1 H), 7.95-8.02 (s, 2H), |
| | | 8.56 (m, 1H, D₂0 exchangeable). |
| | | Mass (m/z): 489.2 (M⁺ + 1). |
| 68 | (S)—S-Methyl(3-(4-(4-3,5-dimethyl | ¹H-NMR (CDCl₃): δ 2.31 (s, 3H), |
| | isoxazole-4-carbonyl)piperazin-1-yl)-3- | 2.35 (s, 3H), 2.44 (s, 3H), 3.05 (bs, |
| | fluorophenyl)-2-oxooxazolidin-5-yl)methylcarbamothioate | 4H), 3.47-3.87 (m, 7H), |
| | | 4.00-4.05 (m, 1H), 4.75-4.78 (m, 1H), |
| | | 5.90 (m, 1H, D₂O exchangeable), |
| | | 6.89-6.94 (m, 1H), 7.09-7.11 (m, |
| | | 1H), 7.44-7.48 (m, 1H). Mass |
| | | (m/z): 493.0 (M⁺ + 1). |
| 69 | (S)—S-Methyl(3-(4-(4-(2-amino-4- | ¹H-NMR (DMSO-d₆): δ 2.21 (s, |
| | (trifluoromethyl)thiazole-5-carbonyl)piperazin- | 3H), 2.96 (bs, 4H), 3.34-3.73 (m, |
| | 1-yl)-3-fluorophenyl)-2-oxo | 7H), 4.07-4.12 (m, 1H), |
| | oxazolidin-5-yl)methylcarbamothioate | 4.69-4.76 (m, 1H), 7.06-7.11 (m, 1H), |
| | | 7.17-7.19 (m, 1H), 7.48-7.51 (m, 1H), |
| | | 7.74 (s, 2H, D₂O exchangeable), |
| | | 8.56 (m, 1H, D₂O exchangeable). |
| | | Mass (m/z): 563.1 (M⁺ + 1). |
| 70 | (S)—S-Methyl (3-(3-fluoro-4-(4-(7-fluoro- | ¹H-NMR (DMSO-d₆): δ 2.21 (s, |
| | 3-methylbenzofuran-2-carbonyl)piperazin- | 3H), 2.38 (s, 3H), 3.05 (bs, 4H), |
| | 1-yl)phenyl)-2-oxooxazolidin- | 3.42-3.47 (m, 4H), 3.70-3.79 (m, |
| | 5-yl)methylcarbamothioate | 3H), 4.08-4.12 (m, 1H), 4.73 (m, |
| | | 1H), 7.12-7.20 (m, 2H), |
| | | 7.33-7.37 (m, 2H), 7.49-7.58 (m, 2H), |
| | | 8.56 (m, 1H, D₂O exchangeable). |
| | | Mass (m/z): 545.1 (M⁺ + 1). |
| 71 | (S)—S-Methyl (3-(3-fluoro-4-(4-(3-methyl | ¹H-NMR (DMSO-d₆): δ 2.20 (s, |
| | isoxazole-4-carbonyl)piperazin-1-yl)phenyl)- | 3H), 2.29 (s, 3H), 3.00 (bs, 4H), |
| | 2-oxooxazolidin-5-yl)methyl | 3.40-3.73 (m, 7H), 4.07-4.12 (m, |
| | carbamothioate | 1H), 4.71-4.74 (m, 1H), |
| | | 7.06-7.11 (m, 1H), 7.18-7.20 (m, 1H), |
| | | 7.48-7.52 (m, 1H), 8.55 (m, 1H, |
| | | D₂O exchangeable), 9.17 (s, 1H). |
| | | Mass (m/z): 478.2 (M⁺ + 1). |
| 72 | (S)—O-Methyl (3-(4-(4-(2-(1H-tetrazol-1- | ¹H-NMR (DMSO-d₆): δ |
| | yl)acetyl)piperazin-1-yl)-3-fluorophenyl)- | 2.99-3.00 (m, 2H), 3.08 (m, 2H), |
| | 2-oxooxazolidin-5-yl)methyl | 3.65-3.69 (m, 4H), 3.77-3.82 (m, 3H) |
| | carbamothioate | 3.88-3.93 (2s (rotational isomers), |
| | | 3H), 4.09-4.15 (m, 1H), |
| | | 4.77-4.90 (2m (rotational isomers), 1H), |
| | | 5.70 (s, 2H), 7.08-7.13 (m, 1H), |
| | | 7.20-7.22 (m, 1H), 7.50-7.54 (m, |
| | | 1H), 9.32 (m, 1H), 9.46-9.54 (2m |
| | | (rotational isomers), 1H, D₂O |
| | | exchangeable). Mass (m/z): |
| | | 478.8 (M⁺). |
| 73 | (S)—O-Methyl(3-(3-fluoro-4-(4-(1- | ¹H-NMR (DMSO-d₆): δ 0.78 (m, |
| | hydroxycyclopropanecarbonyl)piperazin- | 2H), 0.94 (bs, 2H), 2.97 (m, 4H), |
| | 1-yl)phenyl)-2-oxooxazolidin-5-yl)methyl | 3.76-3.77 (m, 4H), 3.79-3.80 (m, |
| | carbamothioate | 3H), 3.88 (2s (rotational isomers), |
| | | 3H), 4.12 (m, 1H), 4.88-4.90 (2m |
| | | (rotational isomers), 1H), 6.39 (s, |
| | | 1H, D₂O exchangeable), 7.10 (m, |
| | | 1H), 7.18 (m, 1H), 7.48-7.52 (m, |
| | | 1H), 9.54 (2m (rotational |
| | | isomers), 1H, D₂O exchangeable). |
| | | Mass (m/z): 452.8 (M⁺). |

### Example 74

### Synthesis of (S)—O-methyl (3-(4-(4-(2-(1,2,3-triazol-1-yl)acetyl)piperazin-1-yl)-3-fluorophenyl)-2-oxooxazolidin-5-yl)methyl carbamothioate

Compound 49 (0.2 g, 0.45 mmol), potassium carbonate (0.11 g, 0.90 mM) and 1H-1,2,3-triazole (0.03 ml, 0.45 mM) were dissolved in DMF (4 ml), stirred well for 15 minutes. The reaction mixture was poured directly into crushed ice, filtered and dried. The crude reaction material was purified by flash chromatography on silica gel using a Biotage SP-1 system (12+M) cartridge using ethyl acetate and hexane as eluent to afford the title compound (0.05 g, yield 21.4%). ¹H-NMR (DMSO-d₆): δ 2.98-3.06 (m, 4H), 3.64-3.69 (m, 4H), 3.75-3.78 (m, 3H), 3.88-3.93 (2 s (rotational isomers), 3H), 4.13 (m, 1H), 4.88 (2 m, (rotational isomers), 1H), 5.55 (s, 2H), 7.11 (m, 1H), 7.19-7.20 (m, 1H), 7.45-7.50 (m, 1H), 7.74 (d, 1H), 8.04 (d, 1H), 9.45-9.54 (2 m (rotational isomers), 1H, D₂O exchangeable). Mass (m/z): 478.1 (M⁺+1).

### Example 75

### Synthesis of (S,E)-O-methyl(3-(3-fluoro-4-(4-(6-(N′-hydroxy carbamimidoyl)nicotinoyl)piperazin-1-yl)phenyl)-2-oxooxazolidin-5-yl)methyl carbamothioate

Compound 8 (0.1 g, 0.20 mmol), hydroxylamine hydrochloride (0.03 g, 0.44 mmol) and triethylamine (0.07 ml, 0.50 mmol) were dissolved in ethanol (3 ml), the reaction mixture was refluxed for 12 hours and the completion of reaction was monitored through TLC using dichloromethane:methanol (9:1), subsequently the reaction mixture was cooled and the precipitated solid was filtered and the residue was washed with ethanol (5 ml) followed by dichloromethane: methanol (1:1) (5 ml) and finally with ethyl acetate:methanol (1:1) mixture (6 ml). The precipitate obtained was dried under reduced pressure to afford the title compound (0.08 g, 75.4%). ¹H-NMR (DMSO-d₆): δ 2.98-3.06 (4H, m), 3.47-3.51 (m, 4H), 3.76-3.81 (m, 3H); 3.88-3.93 (2 s (rotational isomers), 3H), 4.10-4.14 (m, 1H), 4.86-4.90 (2 m, (rotational isomers), 1H); 5.91 (s, 2H, D₂O exchangeable), 7.08-7.12 (m, 1H), 7.18-7.21 (m, 1H), 7.48-7.52 (m, 1H), 7.88-7.94 (m, 2H), 8.84 (s, 1H), 9.46-9.55 (2 m, (rotational isomers), 1H, D₂O exchangeable), 10.10 (1H, s, D₂O exchangeable). Mass (m/z): 532.2 (M⁺+1).

### Example 76

### Synthesis of (S)—O-methyl (3-(4-(4-(1H-imidazole-1-carbonothioyl)piperazin-1-yl)-3-fluorophenyl)-2-oxooxazolidin-5-yl)methyl carbamothioate

To a stirred solution of O-methyl-(5)-N-[3-[3-fluoro-4-[piperazin-1-yl]phenyl]-2-oxo-oxazolidin-5-ylmethyl]thiocarbamate (2.35 g, 6.4 mmol), prepared according to the procedure described in the step 3 of example 1 in DMF (10 ml), was added 1,1′-thiocarbonyl diimidazole (1.37 g, 7.68 mmol) and triethyl amine (3.56 ml, 25.6 mmol) at ambient temperature, and the stirring was continued for another 15 hours. The crude product was partitioned between EtOAc and water, the ethyl acetate layer was then separated, dried over anhydrous Na₂SO₄ and the solvent was removed under vacuum. The crude mass was subjected to column chromatography and the product was eluted using methanol and dichloromethane mixture to afford tht title compound (1.074 g, 35%). ¹H-NMR (DMSO-d₆): δ 3.16 (brs, 4H), 3.4-3.41 (m, 1H), 3.74-3.8 (m, 4H), 3.88 (s, 3H), 3.93 (2 s (rotational isomers), 1H), 4.1-4.12 (m, 1H), 4.10-4.13 (2 m (rotational isomers), 1H), 5.3-5.33 (m, 1H), 7.06-8.07 (m, 6H,), 9.53-9.56 (2 m (rotational isomers), 1H, D₂O exchangeable). Mass (m/z): 479.1 (M⁺+1).

### Antimicrobial Testing

The compounds of invention showed in vitro antibacterial activities when tested by the Agar Dilution Method as specified in documents published by the National Committee for Clinical Laboratory Standards (NCCLS, now CLSI), USA.

Briefly, the compounds of invention were weighed, dissolved in dimethyl sulfoxide, serially two fold diluted in the same solvent and then incorporated into molten Mueller Hinton Agar (MHA) in a petridish just before solidification, with each petridish containing a different concentration of a compound.

The bacterial inoculum was prepared by picking 3 to 5 well isolated bacterial colonies with the same morphological appearance from an 18-24 hours old culture with an inoculating loop, transferring the growth to a tube containing 3 mL of normal saline and adjusting the turbidity of the saline suspension to 0.5 Mc Farland Turbidity Standard equivalent to a bacterial population of 1.5×10⁸ colony forming units (CFU) per mL of suspension. The suspension was diluted 1:10 in saline (i.e. 0.5 mL suspension 4.5 mL saline) to get a bacterial population of 1.5×10⁷ CFU/mL as inoculum.

The bacterial inoculum prepared in the above manner was inoculated onto MHA which had earlier been incorporated with different concentration of the compounds of invention by a Multipoint Inoculator with each inoculum spot containing approximately 1×10⁴ CFU of bacteria.

The inoculated petridishes were incubated at 35° C. in an ambient atmosphere for 16-20 hours. Following incubation, the petridishes were placed on a dark non-reflecting surface and the Minimum Inhibitory Concentration (MIC) was recorded as the concentration that showed no growth of the inoculated culture.

The novel compounds synthesized have shown WC values ranging from <0.03 to >32 μg/ml against gram-positive and gram-negative pathogens including MRSA, PRSP, VRE, M. catarrhalis and H. influenza

The following MIC (μg/ml) were obtained for representative compounds of the invention which are given in the table I:

TABLE I
Antimicrobial Screening (MIC) (μg/ml)

| | Example No | | | | | | | | | |
|---|---|---|---|---|---|---|---|---|---|---|
| Organism | 2 | 40 | 37 | 44 | 45 | 7 | 47 | 76 | 43 | 72 |
| S. aureus MRO 00013* | 1 | 0.25 | 0.5 | 0.5 | 1 | 0.25 | 1 | 0.5 | 0.5 | 1 |
| S. aureus MRO 00055 | 1 | 0.25 | 0.5 | 0.5 | 1 | 0.25 | 1 | 0.5 | 0.5 | 1 |
| S. epidermidis MRO 002046 | 1 | <0.03 | 0.5 | 0.25 | 1 | 0.03 | 0.5 | 0.25 | 0.25 | 0.5 |
| S. aureus MRO 002053 | 1 | 0.06 | 0.25 | 0.5 | 1 | 0.06 | 0.5 | 0.25 | 0.5 | 0.5 |
| S. aureus MRO 0001 | 0.5 | 0.25 | 0.25 | 0.5 | 0.5 | 0.125 | 0.5 | 0.5 | 0.25 | 0.5 |
| S. aureus MRO 0003 | 1 | 0.25 | 0.5 | 0.5 | 1 | 0.25 | 1 | 0.5 | 0.5 | 1 |
| S. aureus MRO 00030 | 1 | 0.125 | 0.5 | 0.5 | 1 | 0.125 | 1 | 0.25 | 0.5 | 0.5 |
| S. aureus MRO 00048 | 0.5 | 0.25 | 0.25 | 0.25 | 0.5 | 0.125 | 0.5 | 0.25 | 0.5 | 0.5 |
| S. aureus MRO 00059 | 1 | 0.25 | 0.5 | 0.5 | 0.5 | 0.25 | 0.5 | 0.25 | 0.5 | 0.5 |
| S. epidermidis MRO 02002 | 1 | 0.125 | 0.5 | 0.5 | 1 | 0.03 | 1 | 0.25 | 0.5 | 0.5 |
| S. epidermidis MRO 02045 | 1 | <0.03 | 0.25 | 0.5 | 1 | 0.125 | 1 | 0.25 | 0.5 | 0.5 |
| S. epidermidis MRO 02095 | 1 | 0.06 | 0.5 | 0.25 | 1 | 0.03 | 0.5 | 0.25 | 0.5 | 0.5 |
| S. saprophyticus MRO 02003 | 1 | 0.06 | 0.5 | 0.5 | 1 | 0.125 | 1 | 0.5 | 0.5 | 1 |
| S. haemolyticus MRO 02064 | 1 | 0.125 | 0.25 | 0.25 | 0.5 | 0.125 | 0.5 | 0.25 | 0.5 | 0.5 |
| E. faecalis MRO 04050 | 1 | 0.25 | 0.25 | 0.5 | 0.5 | 0.125 | 0.5 | 0.25 | 0.25 | 0.5 |
| E. faecalis MRO 04205 | 0.5 | 0.25 | 0.25 | 0.25 | 0.5 | 0.125 | 0.5 | 0.25 | 0.25 | 0.5 |
| E. faecalis MRO 04035 | 0.5 | 0.25 | 0.25 | 0.25 | 0.5 | 0.125 | 0.5 | 0.25 | 0.25 | 0.5 |
| E. faecium MRO 04198 | 0.5 | 0.25 | 0.25 | 0.25 | 0.5 | 0.25 | 0.5 | 0.25 | 0.5 | 0.5 |
| E. faecium MRO 04037 | 0.5 | 0.25 | 0.25 | 0.25 | 0.5 | 0.25 | 0.5 | 0.25 | 0.25 | 0.5 |
| E. faecium MRO 04038 | 0.5 | 0.25 | 0.25 | 0.25 | 0.5 | 0.125 | 0.5 | 0.25 | 0.25 | 0.5 |
| E. faecalis ATCC 51299 | 0.5 | 0.25 | 0.25 | 0.25 | 0.5 | 0.125 | 0.5 | 0.25 | 0.25 | 0.5 |
| E. faecium ATCC 700221 | 0.5 | 0.125 | 0.25 | 0.25 | 0.5 | 0.125 | 0.5 | 0.25 | 0.25 | 0.5 |
| E. faecalis ATCC 29212 | 1 | 0.25 | 0.25 | 0.25 | 0.5 | 0.125 | 0.5 | 0.25 | 0.25 | 0.5 |
| S. aureus ATCC 29213 | 1 | 0.25 | 0.5 | 0.5 | 1 | 0.125 | 1 | 0.5 | 0.5 | 1 |
| S. aureus ATCC 43300 | 1 | 0.125 | 0.5 | 0.5 | 0.5 | 0.125 | 0.5 | 0.25 | 0.5 | 1 |
| M. catarrhalis ATCC 43617 | 1 | 0.06 | 1 | 1 | 0.5 | 0.25 | 1 | 1 | 1 | 2 |
| M. catarrhalis ATCC 43627 | 4 | 0.125 | 2 | 2 | 2 | 0.25 | 2 | 1 | 2 | 2 |
| M. catarrhalis ATCC 43628 | 4 | 0.125 | 1 | 1 | 1 | 0.25 | 2 | 1 | 2 | 2 |

MRO—Microbial Resource Orchid
1) S. aureus—Staphylococus aureus
2) E. faecalis—Enterococcus faecalis
3) .E. faecium—Enterococcus faecium
4) M. catarrhalis—Moraxella catarrhalis
ATCC—American Type Culture Collection

We claim:

1. Novel compounds of the formula (I) 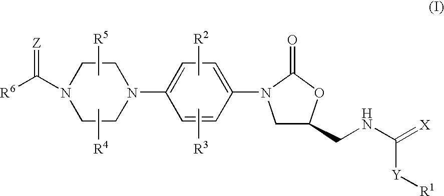
their derivatives, analogs, tautomeric forms, stereoisomers, polymorphs, solvates, pharmaceutically acceptable salts and compositions metabolites and prodrugs, wherein, suitable groups represented by R¹ are selected from hydrogen; (C₁-C₆)allyl groups comprising methyl, ethyl, n-propyl, isopropyl, n-butyl, isobutyl, t-butyl, n-pentyl, isopentyl and hexyl, which are optionally substituted; haloalkyl groups comprising chloromethyl, chloroethyl, trifluoromethyl, trifluoroethyl, dichloromethyl and dichloroethyl, which are optionally substituted; (C₃-C₆)cycloalkyl groups comprising cyclopropyl, cyclobutyl, cyclopentyl and cyclohexyl, which are optionally substituted; alkylamine groups comprising NH₂CH₂—, NH₂C₃H₆— and NH₂C₆H₁₂—, which are optionally substituted; aryl groups comprising phenyl and naphthyl, which are optionally substituted; aralkyl groups comprising phenylmethyl, phenylethyl, naphthylmethyl and naphthylethyl, which are optionally substituted; heterocyclyl groups comprising pyrrolidinyl, morpholinyl, thiomorpholinyl, piperidinyl and piperazinyl, which are optionally substituted; heteroaryl groups comprising pyridyl, thienyl, furyl, pyrrolyl, oxazolyl, thiazolyl, imidazolyl, isooxazolyl, oxadiazolyl, triazolyl, thiadiazolyl, tetrazolyl, pyrimidinyl, pyrazinyl, pyridazinyl, benzopyranyl, benzofuranyl, benzimidazolyl, benzoxazolyl, benzothiazolyl, benzopyrrolyl, benzoxadiazolyl and benzothiadiazolyl, which are optionally substituted; alkylsulfonyl, arylsulfonyl and aralkylsulfonyl, which are optionally substituted;
suitable groups represented by R² and R³ are selected from hydrogen; halogen atoms comprising fluorine, chlorine, bromine or iodine; hydroxyl, (C₁-C₆) alkyl groups comprising methyl, ethyl, n-propyl, isopropyl, n-butyl, isobutyl, t-butyl, n-pentyl, isopentyl and hexyl, which are optionally substituted; (C₁-C₆) alkoxy groups comprising methoxy, ethoxy, n-propoxy and isopropoxy, which are optionally substituted;
suitable groups represented by R⁴ and R⁵ are selected from hydrogen; cyano; nitro; amino; halogen; hydroxyl; (C₁-C₆) alkyl groups comprising methyl, ethyl, n-propyl, isopropyl, n-butyl, isobutyl, t-butyl, n-pentyl, isopentyl, hexyl, which are optionally substituted; haloalkyl groups comprising chloromethyl, chloroethyl, trifluoromethyl, trifluoroethyl, dichloromethyl and dichloroethyl, which are optionally substituted; (C₁-C₆) alkoxy groups comprising methoxy, ethoxy, n-propoxy and isopropoxy, which are optionally substituted; (C₁-C₆)alkylthio groups comprising methylthio, ethylthio, n-propylthio and iso-propylthio, which are optionally substituted; (C₃-C₆)cycloalkyl groups comprising cyclopropyl, cyclobutyl, cyclopentyl and cyclohexyl, which are optionally substituted or either of R⁴ or R⁵ represent an oxo or thioxo group; substituted or unsubstituted benzyl group;
suitable groups represented by R⁶ are selected from a (C₁-C₆) alkyl groups comprising methyl, ethyl, n-propyl, isopropyl, n-butyl, isobutyl, t-butyl, n-pentyl, isopentyl and hexyl, which are optionally substituted; mono or dihydroxyalkyloxymethyl groups comprising HO—CH₂—CH₂—O—CH₂— and HO—CH₂—CH(OH)—CH₂—O—CH₂—, haloalkyl groups comprising chloromethyl, chloroethyl, trifluoromethyl, trifluoroethyl, dichloromethyl and dichloroethyl, which are optionally substituted; (C₃-C₆) cycloalkyl groups comprising cyclopropyl, cyclobutyl, cyclopentyl, and cyclohexyl, which are optionally substituted; alkylamine groups comprising NH₂CH₂—, NH₂C₂H₄—, NH₂C₃H₆— and NH₂C₆H₁₂—, which are optionally substituted; aryl groups comprising phenyl and naphthyl, which are optionally substituted; aralkyl groups comprising phenylmethyl, phenylethyl, naphthylmethyl and naphthylethyl, which are optionally substituted; arylalkenyl groups comprising benzene-prop-1-enyl, which are optionally substituted; benzyloxyalkyl groups comprising benzyloxymethyl, benzyloxyethyl and benzyloxypropyl, which are optionally substituted; heteroaryl groups comprising pyridyl, thienyl, furyl, pyrrolyl, oxazolyl, thiazolyl, imidazolyl, isooxazolyl, oxadiazolyl, triazolyl, thiadiazolyl, tetrazolyl, pyrimidinyl, pyrazinyl, pyridazinyl, quinolinyl, quinoxalinyl, indolyl, indazolyl, benzopyranyl, benzofuranyl, benzimidazolyl, benzoxazolyl, benzothiazolyl, benzopyrrolyl, benzoxadiazolyl and benzothiadiazolyl, which are optionally substituted; heteroaralkyl groups comprising pyridylmethyl, indolylmethyl, triazolylmethyl and tetrazolylmethyl, which are optionally substituted; heteroarylalkenyl groups comprising furan-ethen-1-yl and furan-prop-1-enyl which are optionally substituted; heterocyclyl groups comprising pyrrolidinyl, morpholinyl, thiomorpholinyl, thiazolidinyl, piperidinyl and piperazinyl, which are optionally substituted; substituted or unsubstituted groups selected from TR⁸, wherein T represents O or S; R⁸ represents substituted or unsubstituted groups selected from (C₁-C₆) alkyl groups comprising methyl, ethyl, n-propyl, isopropyl, n-butyl, isobutyl, t-butyl, n-pentyl, isopentyl and hexyl, which are optionally substituted; haloalkyl groups comprising chloromethyl, chloroethyl, trifluoromethyl, trifluoroethyl, dichloromethyl and dichloroethyl, which are optionally substituted; (C₃-C₆) cycloalkyl groups comprising cyclopropyl, cyclobutyl, cyclopentyl and cyclohexyl, which are optionally substituted; alkylamine groups comprising NH₂CF₁₂—, NH₂C₂H₄—, NH₂C₃H₆— and NH₂C₆H₁₂— which are optionally substituted; aryl groups comprising phenyl and naphthyl, which are optionally substituted; aralkyl groups comprising phenylmethyl, phenylethyl, naphthylmethyl and naphthylethyl, which are optionally substituted; heterocyclyl groups comprising pyrrolidinyl, morpholinyl, thiomorpholinyl, piperidinyl and piperazinyl, which are optionally substituted; heteroaryl groups comprising pyridyl, thienyl, furyl, pyrrolyl, oxazolyl, thiazolyl, imidazolyl, isooxazolyl, oxadiazolyl, triazolyl, thiadiazolyl, tetrazolyl, pyrimidinyl, pyrazinyl, pyridazinyl, benzopyranyl, benzofuranyl, benzimidazolyl, benzoxazolyl, benzothiazolyl, benzopyrrolyl, benzoxadiazolyl and benzothiadiazolyl, which are optionally substituted; alkylsulfonyl; arylsulfonyl and aralkylsulfonyl, which are optionally substituted;
wherein X and Y independently represent oxygen or sulfur; Z represent oxygen or sulphur or N(O)ₙR⁷, wherein suitable groups represented by R⁷ are selected from hydrogen; hydroxyl, substituted or unsubstituted linear or branched (C₁-C₆)alkyl groups comprising methyl, ethyl, n-propyl, isopropyl, n-butyl, isobutyl, t-butyl, n-pentyl and hexyl, which are optionally substituted; (C₃-C₆) cycloalkyl groups comprising cyclopropyl, cyclobutyl, cyclopentyl and cyclohexyl, which are optionally substituted;
haloalkyl groups comprising chloromethyl, chloroethyl, trifluoromethyl, trifluoroethyl, dichloromethyl and dichloroethyl, which are optionally substituted; aryl groups comprising phenyl and naphthyl, which are optionally substituted; aralkyl groups comprising phenylmethyl, phenylethyl, naphthylmethyl and naphthylethyl, which are optionally substituted; acyl groups comprising —C(═O)CH₃, —C(═O)C₂H₅, —C(═O)C₃H₇, —C(═O)C₆H₁₃ and benzoyl, which are optionally substituted; thioacyl groups comprising —C(═S)CH₃, —C(═S)C₂H₅, —C(═S)C₃H₇ and —C(═S)C₆H₁₃, which are optionally substituted; alkylsulfonyl groups comprising methylsulfonyl, ethylsulfonyl, n-propylsulfonyl and iso-propylsulfonyl, which are optionally substituted; arylsulfonyl groups comprising phenylsulfonyl, naphthylsulfonyl, which are optionally substituted; aralkylsulfonyl groups comprising phenylmethylsulfonyl, phenylethylsulfonyl, naphthylmethylsulfonyl and naphthylethylsulfonyl, which are optionally substituted; heteroaryl groups comprising pyridyl, thienyl, furyl, pyrrolyl, oxazolyl, thiazolyl, imidazolyl, isooxazolyl, oxadiazolyl, triazolyl, thiadiazolyl, tetrazolyl, pyrimidinyl, pyrazinyl, pyridazinyl, benzopyranyl, benzofuranyl, benzimidazolyl, benzoxazolyl, benzothiazolyl, benzopyrrolyl, benzoxadiazolyl and benzothiadiazolyl, which are optionally substituted; heterocyclyl groups comprising pyrrolidinyl, morpholinyl, thiomorpholinyl, piperidinyl and piperazinyl, which are optionally substituted;
n is an integer of 0 or 1;
the substituents on any of the groups represented by R¹, R², R³, R⁴, R⁵, R⁶, R⁷ and R⁸ are selected from one or more groups represented by halogen; hydroxy; formyl; nitro; cyano; azido; amino; alkyl; aryl; alkylamino; alkylsulfonylamino; aralkoxy; arylthio; alkylaminocarbonyl; haloalkyl; alkylthio; acylamino; alkoxy; acyl; cycloalkylacyl; heteroarylacyl; N-hydroxyimidamide; carboxylic acid and its derivatives comprising esters and amides and these substituents are as defined above; the above substituents, which in turn are further substituted by the groups selected from alkyl; halogen; amino; nitro and hydroxy.

2. Novel compounds of claim 1, selected from a group consisting of
(S)—O-Methyl(3-(4-(4-(benzofuran-2-carbonyl)piperazin-1-yl)-3-fluorophenyl)-2-oxooxazolidin-5-yl)methylcarbamothioate;
(S)—O-Methyl(3-(3-fluoro-4-(4-nicotinoylpiperazin-1-yl)phenyl)-2-oxooxazolidin-5-yl)methylcarbamothioate;
(S)—O-Methyl(3-(3-fluoro-4-(4-(pyrazine-2-carbonyl)piperazin-1-yl)phenyl)-2-oxo oxazolidin-5-yl)methylcarbamothioate;
(S)—O-Methyl(3-(3-fluoro-4-(4-(3,4,5-trimethoxybenzoylpiperazin-1-yl)phenyl)-2-oxo oxazolidin-5-yl)methylcarbamothioate;
(S)—O-Methyl(3-(4-(4-(4-acetamidobenzoyl)piperazin-1-yl)-3-fluorophenyl)-2-oxo oxazolidin-5-yl)methylcarbamothioate;
(S)—O-Methyl (3-(3-fluoro-4-(4-(5-methylpyrazine-2-carbonyl)piperazin-1-yl)phenyl)-2-oxooxazolidin-5-yl)methylcarbamothioate;
(S)—O-Methyl (3-(3-fluoro-4-(4-(5-nitrofuran-2-carbonyl)piperazin-1-yl)phenyl)-2-oxooxazolidin-5-yl)methylcarbamothioate;
(S)—O-Methyl(3-(4-(4-(6-cyanonicotinoyl)piperazin-1-yl)-3-fluorophenyl)-2-oxo oxazolidin-5-yl)methylcarbamothioate;
(S)—O-Methyl (3-(3-fluoro-4-(4-(1-methyl-pyrrole-2-carbonyl)piperazin-1-yl)phenyl)-2-oxooxazolidin-5-yl)methylcarbamothioate;
(S)—O-Methyl(3-(3-fluoro-4-(4-isonicotinoylpiperazin-1-yl)phenyl)-2-oxooxazolidin-5-yl)methylcarbamothioate;
(S)—O-Methyl(3-(3-fluoro-4-(4-(quinoline-3-carbonyl)piperazin-1-yl)phenyl)-2-oxo oxazolidin-5-yl)methylcarbamothioate;
(S)—O-Methyl(3-(4-(4-(6-chloronicotinoyl)piperazin-1-yl-3-fluorophenyl)-2-oxo oxazolidin-5-yl)methylcarbamothioate;
(S)—O-Methyl (3-(4-(4-(1H-indole-2-carbonyl)piperazin-1-yl)-3-fluorophenyl)-2-oxooxazolidin-5-yl)methylcarbamothioate;
(S)—O-Methyl(3-(3-fluoro-4-(4-(2-fluoronicotinoyl)piperazin-1-yl)phenyl)-2-oxo oxazolidin-5-yl)methylcarbamothioate;
(S)—O-Methyl (3-(3-fluoro-4-(4-(5-fluoro-1,4-indole-2-carbonyl)piperazin-1-yl)phenyl)-2-oxooxazolidin-5-yl)methylcarbamothioate;
(S)—O-Methyl(3-(4-(4-(4-chloronicotinoyl)piperazin-1-yl)-3-fluorophenyl)-2-oxo oxazolidin-5-yl)methylcarbamothioate;
(S)—O-Methyl(3-(3-fluoro-4-(4-(6-methylnicotinoyl)piperazin-1-yl)phenyl)-2-oxooxazolidin-5-yl)methylcarbamothioate;
(S)—O-Methyl(3-(4-(4-(1H-imidazole-4-carbonyl)piperazin-1-yl)-3-fluorophenyl)-2-oxo oxazolidin-5-yl)methylcarbamothioate;
(S)—O-Methyl (3-(4-(4-(1-cyanocyclopropanecarbonyl)piperazin-1-yl)-3-fluorophenyl)-2-oxooxazolidin-5-yl)methylcarbamothioate;
(S)—O-Methyl (3-(4-(4-(5-bromonicotinoyl)piperazin-1-yl)-3-fluorophenyl)-2-oxooxazolidin-5-yl)methylcarbamothioate;
(S)—O-Methyl(3-(3-fluoro-4-(4-(quinoxaline-2-carbonyl)piperazin-1-yl)phenyl)-2-oxooxazolidin-5-yl)methylcarbamothioate;
(S)—O-Methyl (3-(3-fluoro-4-(4-(6-(trifluoromethypnicotinoyl)piperazin-1-yl)phenyl)-2-oxooxazolidin-5-yl)methylcarbamothioate;
(S)—O-Methyl(3-(3-fluoro-4-(4-(1-p-tolylcyclopropanecarbonyl)piperazin-1-yl)phenyl)-2-oxooxazolidin-5-yl)methylcarbamothioate;
(S)—O-Methyl(3-(4-(4-(2,6-dichloro-5-fluoronicotinoyl)piperazin-1-yl)-3-fluorophenyl)-2-oxooxazolidin-5-yl)methylcarbamothioate;
(S)—O-Methyl(3-(4-(4-(1H-indazole-3-carbonyl)piperazin-1-yl)-3-fluorophenyl)-2-oxooxazolidin-5-yl)methylcarbamothioate;
(S)—O-Methyl(3-(3-fluoro-4-(4-(6-hydroxynicotinoyl)piperazin-1-yl)phenyl)-2-oxo oxazolidin-5-yl)methylcarbamothioate;
(S)—O-Methyl (3-(4-(4-(benzo[d]thiazole-6-carbonyl)piperazin-1-yl)-3-fluorophenyl)-2-oxooxazolidin-5-yl)methylcarbamothioate;
(S)—O-Methyl (3-(3-fluoro-4-(4-(thiazolidine-2-carbonyl)piperazin-1-yl)phenyl)-2-oxooxazolidin-5-yl)methylcarbamothioate;
(S)—O-Methyl (3-(4-(4-(1H-benzo[d]imidazole-5-carbonyl)piperazin-1-yl)-3-fluorophenyl)-2-oxooxazolidin-5-yl)methylcarbamothioate;
(S)—O-Methyl(3-(4-(4-(5-chlorobenzofuran-2-carbonyl)piperazin-1-yl)-3-fluorophenyl)-2-oxooxazolidin-5-yl)methylcarbamothioate;
(S)—O-Methyl(3-(3-fluoro-4-(4-((R)-thiazolidine-4-carbonyl)piperazin-1-yl)phenyl)-2-oxooxazolidin-5-yl)methylcarbamothioate;
(S)—O-Methyl(3-(3-fluoro-4-(4-(2,2,2-trifluoroacetyl)piperazin-1-yl)phenyl)-2-oxo oxazolidin-5-yl)methylcarbamothioate;
(S)—O-Methyl (3-(4-(4-(2,2-difluorocyclopropanecarbonyl)piperazin-1-yl)-3-fluoro phenyl)-2-oxooxazolidin-5-yl)methylcarbamothioate;
(S)—O-Methyl(3-(4-(4-(4-aminonicotinoyl)piperazin-1-yl)-3-fluorophenyl)-2-oxooxazolidin-5-yl)methylcarbamothioate;
(S)—O-Methyl (3-(3-fluoro-4-(4-(6-fluoronicotinoyl)piperazin-1-yl)phenyl)-2-oxo oxazolidin-5-yl)methylcarbamothioate;
(S)—O-Methyl(3-(4-(4-(2-amino-4-(trifluoromethyl)thiazole-5-carbonyl)piperazin-1-yl)-3-fluorophenyl)-2-oxooxazolidin-5-yl)methylcarbamothioate;
(S)—O-Methyl(3-(4-(4-(6-aminonicotinoyl)piperazin-1-yl)-3-fluorophenyl)-2-oxo oxazolidin-5-yl)methylcarbamothioate;
(S)—O-Methyl(3-(3-fluoro-4-(4-(5-fluoronicotinoyl)piperazin-1-yl)phenyl)-2-oxo oxazolidin-5-yl)methylcarbamothioate;
(S)—O-Methyl (3-(3-fluoro-4-(4-(5-methylisoxazole-3-carbonyl)piperazin-1-yl)phenyl)-2-oxooxazolidin-5-yl)methylcarbamothioate;
(S)—O-Methyl(3-(3-fluoro-4-(4-(3-(5-nitrofuran-2-yl)acryloyl)piperazin-1-yl)phenyl)-2-oxooxazolidin-5-yl)methylcarbamothioate;
(S)—O-Methyl(3-(4-(4-(2-aminoacetyl)piperazin-1-yl)-3-fluorophenyl)-2-oxooxazolidin-5-yl)methylcarbamothioate hydrochloride;
(S)—O-Methyl(3-(4-(4-((S)-2-amino-3-hydroxypropanoyl)piperazin-1-yl)-3-fluoro phenyl)-2-oxooxazolidin-5-yl)methylcarbamothioate hydrochloride;
(S)—O-Methyl(3-(3-fluoro-4-(4-(2-(methylsulfonamido) acetyl)piperazin-1-yl)phenyl)-2-oxooxazolidin-5-yl)methylcarbamothioate;
(S)—O-Methyl (3-(3-fluoro-4-(4-(isoxazole-5-carbonyl)piperazin-1-yl)phenyl)-2-oxooxazolidin-5-yl)methylcarbamothioate;
(S)—O-Methyl (3-(4-(4-(2-(benzyloxy)acetyl)piperazin-1-yl)-3-fluorophenyl)-2-oxooxazolidin-5-yl)methylcarbamothioate;
(S)—O-Methyl(3-(4-(4-(cyoclohexanecarbonyl)piperazin-1-yl)-3-fluorophenyl)-2-oxo oxazolidin-5-yl)methylcarbamothioate;
(S)—O-Methyl(3-(4-(4-(cyclopropanecarbonyl)piperazin-1-yl)-3-fluorophenyl)-2-oxo oxazolidin-5-yl)methylcarbamothioate;
(S)—O-Methyl(3-(4-(4-(cyclopentanecarbonyl)piperazin-1-yl)-3-fluorophenyl)-2-oxooxazolidin-5-yl)methylcarbamothioate;
(S)—O-Methyl(3-(4-(4-(2-chloroacetyl)piperazin-1-yl)-3-fluorophenyl)-2-oxooxazolidin-5-yl)methylcarbamothioate;
(S)—S-Methyl(3-(4-(4-(4-chlorobenzoyl)piperazin-1-yl)-3-fluorophenyl)-2-oxooxazolidin-5-yl)methylcarbamothioate;
(S)—S-Methyl(3-(3-fluoro-4-(4-(morpholine-4-carbonyl)piperazin-1-yl)phenyl)-2-oxo oxazolidin-5-yl)methylcarbamothioate;
(S)—S-Methyl(3-(3-fluoro-4-(4-(4-fluorobenzoyl)piperazin-1-yl)phenyl)-2-oxo oxazolidin-5-yl)methylcarbamothioate;
(S)—S-Methyl(3-(4-(4-(2-chloroacetyl)piperazin-1-yl)-3-fluorophenyl)-2-oxooxazolidin-5-yl)methylcarbamothioate;
(S)—S-Methyl(3-(4-(4-(3-chlorobenzoyl)piperazin-1-yl)-3-fluorophenyl)-2-oxooxazolidin-5-yl)methylcarbamothioate;
(S)—S-Methyl(3-(3-fluoro-4-(4-pivaloylpiperazin-1-yl)phenyl)-2-oxooxazolidin-5-yl)methylcarbamothioate;
(S)—S-Methyl(3-(3-fluoro-4-(4-(morpholine-4-carbonothioyl)piperazin-1-yl)phenyl)-2-oxooxazolidin-5-yl)methylcarbamothioate;
(S)—S-Methyl(3-(4-(4-(2-(2,5-dichlorophenylthio)acetyl)piperazin-1-yl)-3-fluorophenyl)-2-oxooxazolidin-5-yl)methylcarbamothioate;
(S)—S-Methyl(3-(4-(4-(imidazole-1-carbonyl)piperazin-1-yl)-3-fluorophenyl)-2-oxooxazolidin-5-yl)methylcarbamothioate;
(S)—S-Methyl(3-(3-fluoro-4-(4-(1-methyl-indazole-3-carbonyl)piperazin-1-yl)phenyl)-2-oxooxazolidin-5-yl)methylcarbamothioate;
(S)—S-Methyl(3-(4-(4-(2-cyanoacetyl)piperazin-1-yl)-3-fluorophenyl)-2-oxooxazolidin-5-yl)methylcarbamothioate;
(S)—S-Methyl(3-(4-(4-(5-chlorothiophene-2-carbonyl)piperazin-1-yl)-3-fluorophenyl)-2-oxooxazolidin-5-yl)methylcarbamothioate;
(S)—S-Methyl(3-(4-(4-(2-aminonicotinoyl)piperazin-1-yl)-3-fluorophenyl)-2-oxo oxazolidin-5-yl)methylcarbamothioate;
(S)—S-Methyl(3-(3-fluoro-4-(4-(6-fluoro-2-naphthoyl)piperazin-1-yl)phenyl)-2-oxo oxazolidin-5-yl)methylcarbamothioate;
(S)—S-Methyl(3-(4-(4-(2,2-difluorocyclopropanecarbonyl)piperazin-1-yl)-3-fluoro phenyl)-2-oxooxazolidin-5-yl)methylcarbamothioate;
(S)—S-Methyl(3-(3-fluoro-4-(4-(6-fluoropicolinoyl)piperazin-1-yl)phenyl)-2-oxooxazolidin-5-yl)methylcarbamothioate;
(S)—S-Methyl(3-(3-fluoro-4-(4-(5-methylisoxazole-3-carbonyl)piperazin-1-yl)phenyl)-2-oxooxazolidin-5-yl)methylcarbamothioate;
(S)—S-Methyl(3-(4-(4-aminonicotinoyl)piperazin-1-yl)-3-fluorophenyl)-2-oxooxazolidin-5-yl)methylcarbamothioate;
(S)—S-Methyl(3-(4-(4-(3,5-dimethylisoxazole-4-carbonyl)piperazin-1-yl)-3-fluorophenyl)-2-oxooxazolidin-5-yl)methylcarbamothioate;
(S)—S-Methyl (3-(4-(4-(2-amino-4-(trifluoromethyl) thiazole-5-carbonyl)piperazin-1-yl)-3-fluorophenyl)-2-oxooxazolidin-5-yl)methylcarbamothioate;
(S)—S-Methyl(3-(3-fluoro-4-(4-(7-fluoro-3-methylbenzofuran-2-carbonyl)piperazin-1-yl)phenyl)-2-oxooxazolidin-5-yl)methyl carbamothioate;
(S)—S-Methyl(3-(3-fluoro-4-(4-(3-methylisoxazole-4-carbonyl)piperazin-1-yl)phenyl)-2-oxooxazolidin-5-yl)methylcarbamothioate;
(S)—O-Methyl (3-(4-(4-(2-(1H-tetrazol-1-yl)acetyl)piperazin-1-yl)-3-fluorophenyl)-2-oxooxazolidin-5-yl)methylcarbamothioate;
(S)—O-Methyl (3-(3-fluoro-4-(4-(1-hydroxycyclopropanecarbonyl)piperazin-1-yl)phenyl)-2-oxooxazolidin-5-yl)methyl carbamothioate;
(S)—O-Methyl(3-(4-(4-(2-(1,2,3-triazol-1-yl)acetyl)piperazin-1-yl)-3-fluorophenyl)-2-oxooxazolidin-5-yl)methylcarbamothioate;
(S,E)-O-Methyl(3-(3-fluoro-4-(4-(6-(N′-hydroxycarbamimidoyl)nicotinoyl)piperazin-1-yl)phenyl)-2-oxooxazolidin-5-yl)methylcarbamothioate;
(S)—O-Methyl (3-(4-(4-(1H-imidazole-1-carbonothioyl)piperazin-1-yl)-3-fluorophenyl)-2-oxooxazolidin-5-yl)methylcarbamothioate;
(S)—O-Methyl(3-(3-fluoro-4-(4-(2-(2-hydroxyethoxy)acetyl)piperazin-1-yl)phenyl)-2-oxooxazolidin-5-yl)methylcarbamothioate and
(S)—O-Methyl (3-(4-(4-(2-(2,3-dihydroxypropoxy)acetyl)piperazin-1-yl)-3-fluorophenyl)-2-oxooxazolidin-5-yl)methylcarbamothioate.

3. A process for the preparation of compounds of the formula (I), as claimed in claim 1, which comprises:
reacting a compound of formula (VIII) with a compound of formula (IX) 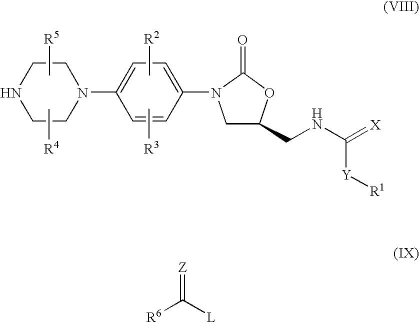
wherein L is a leaving group and all the other symbols are as defined earlier, to produce the compound of formula (I), using appropriate reagents selected from dicyclohexylcarbodiimide (DCC), 1-ethyl-3-(3-dimethylaminopropyl)carbodiimide (EDAC), benzotriazole-1-yl-oxy-tris-(dimethylamino)-phosphonium hexafluoro phosphate (BOP), N-hydroxysuccinimide (NHS), 1-hydroxybenztriazole hydrate (HOBt), carried out using appropriate solvents selected from dimethyl formamide and THF in the presence of appropriate organic or inorganic bases selected from triethylamine, diisopropylethylamine and potassium carbonate.

4. A process for the preparation of compounds of the formula (I), as claimed in claim 1, wherein R₆ is aminomethyl and all the other symbols are as defined earlier, which comprises: 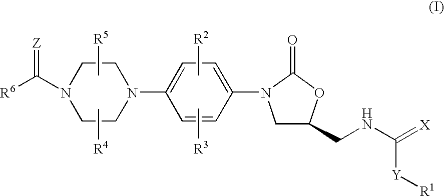
reacting BOC protected glycine with a compound of formula (VIII), followed by deprotection using appropriate reagents selected from trifluoroacetic acid and dioxane/HCl and subsequent derivatization using mesyl chloride.

5. A process for the preparation of compounds of the formula (I), as claimed in claim 1, wherein R₆ is 6-(N′-hydroxycarbamimidoyl)nicotinoyl and all the other symbols are as defined earlier, which comprises: 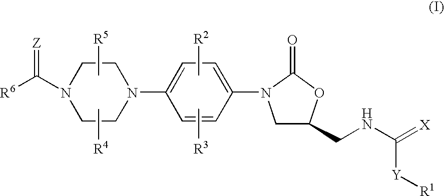
reacting a compound of formula (I) wherein R₆ is 6-cyanonicotinoyl, with hydroxylamine hydrochloride and an appropriate base selected from potassium carbonate and triethyl amine under reflux conditions.

6. A pharmaceutical composition comprising a compound of formula (I) according to claim 1, as an active ingredient along with a pharmaceutically acceptable carrier, diluent, excipient or solvate.

7. The pharmaceutical composition according to claim 1, wherein the composition is in a form of a tablet, capsule, powder, syrup, solution, aerosol or suspension.

8. The pharmaceutical composition as claimed in claim 1, wherein the amount of the compound of formula (I) in the composition is less than 70% by weight.

9. A method of prophylaxis or treatment of the bacterial infection in a mammal, comprising administering an effective amount of a compound of claim 1 to the mammal in need thereof.

10. A method of prophylaxis or treatment of the bacterial infection in a mammal, comprising administering an effective amount of a compound of claim 2 to the mammal in need thereof.
